(19) United States
(12) Patent Application Publication
(10) Pub. No.: US 2007/0087344 A1
(43) Pub. Date: Apr. 19, 2007

(54) Methods for characterizing signaling pathways and compounds that interact therewith

(75) Inventors: Ivan Plavec, Sunnyvale, CA (US); Ellen L. Berg, Palo Alto, CA (US)

Correspondence Address: BOZICEVIC, FIELD & FRANCIS LLP, 1900 UNIVERSITY AVENUE, SUITE 200, EAST PALO ALTO, CA, 94303, US

(73) Assignee: BIOSEEK, INC.

(21) Appl. No.: 10/554,043

(22) PCT Filed: Apr. 23, 2004
(86) PCT No.: PCT/US04/12449

§ 371 Date: Aug. 10, 2006

Related U.S. Application Data
(60) Provisional application No. 60/465,152, filed on Apr. 23, 2003.

(51) Int. Cl. C12Q 1/68; G06F 19/00

(52) U.S. Cl. 435/6; 702/20

(57) ABSTRACT

The genes in a signaling pathway can be identified by over or under-expressing each member of a set of genes to be grouped in signaling pathways and measuring parameters under stimulatory conditions. Genes in a common signaling pathway give similar parameter profiles under such conditions. Moreover, the interactions between signaling pathways and the genes and gene products that mediate those interactions can be determined by measuring the response of those pathways to stimuli under conditions that reflect a complex cellular environment. The order in which genes and their products transmit signals through signaling pathways can be determined by assaying the effects of activators and inhibitors on those genes and gene products, and the mechanism of action of a compound of interest can be determined by comparing parameter profiles generated by the compound of interest to those generated with compounds of known mechanism of action.

### FIELD OF THE INVENTION

The present invention relates generally to the analysis of gene function and the identification of signaling pathways, and more particularly to methods for characterizing signaling pathway architecture, finding relationships between signaling components, and identifying drug targets and the mechanisms of drug action. The invention therefore relates to the fields of biology, molecular biology, chemistry, medicinal chemistry, pharmacology, and medicine.

### BACKGROUND OF THE INVENTION

Knowledge of the biochemical pathways by which cells detect and respond to stimuli is important for the discovery, development, and correct application of pharmaceutical products. Such pathways are called “signaling pathways.” Most current methods for elucidating all of the gene products in a signaling pathway require prior knowledge of at least one gene or gene product (sometimes called a “member” or “component”) of the pathway.

Such methods include, for example, protein-protein interaction assays (including yeast two-hybrid and immunoprecipitation-based methods), which can be used to identify proteins that directly bind to each other, and so are presumably functionally involved in signaling. In these methods, one starts with a known protein, such as a receptor molecule, and tries to identify proteins that bind specifically to an intracellular or extracellular portion of the receptor. A number of such proteins (most commonly called receptor associated proteins) have been described.

Specific, inhibitors of certain genes and gene products can also be used to determine if a particular gene plays a role in a signaling pathway. Most commonly, there is a specific assay, such as, for example, an assay based on TNF-alpha-induced expression of ICAM, and if specific inhibition of a gene or gene product results in detectable reduction in assay output, then one concludes that the particular gene or gene product plays a role in the signaling pathway of interest.

Knowledge of the biochemical pathways by which cells detect and respond to stimuli is important for the discovery, development, and correct application of pharmaceutical products. Cellular physiology involves multiple pathways, which have complex relationships. For example, pathways split and join; there are redundancies in performing specific actions; and response to a change in one pathway can modify the activity of another pathway, both within and between cells. In order to understand how a candidate agent is acting and whether it will have the desired effect, the end result, and effect on pathways of interest is as important as knowing the target protein.

BioMAP® methods of analysis for determining the pathways affected by an agent or genotype modification in a cell, and for identifying common modes of operation between agents and genotype modifications, are described in U.S. Pat. No. 6,656,695; and International applications WO01/067103 and WO03/023573. Cells capable of responding to factors, simulating a state of interest are employed. A sufficient number of factors are employed to involve a plurality of pathways and a sufficient number of parameters are selected to provide an informative dataset. The data resulting from the assays can be processed to provide robust comparisons between different environments and agents.

While these methods enable the identification of the genes and gene products in a signaling pathway, there remains a need for methods to determine the order of the components in the pathway, as well as for methods to identify pathways that interact with one another and the component(s) that mediate such interactions. Moreover, methods are needed to identify the components in a pathway that are the targets of action of a drug, including not only the primary target by which a drug mediates its beneficial effects but also secondary targets that contribute to an undesired side-effect profile. The present invention meets these and other needs.

### SUMMARY OF THE INVENTION

The present invention provides methods for analysis of interactions between polypeptides in a signaling pathway, where the associations may comprise physical and/or functional relationships. In these methods, the consequences, or biological responses, that result from activation and inhibition at various steps along pathways are measured and used to determine whether genes are in a common signaling pathway or at an intersection of two different signaling pathways; the order of action of the various components of the pathways; and the mechanism of action of a compound that affects a signaling pathway.

In one embodiment, the components of a signaling pathway are determined by exposing a set of recombinant cells, each member of which over or under-expresses a gene to be identified either as a gene in the pathway or not in the pathway, to a variety of biologically active factors that are either activators or inhibitors of signaling pathways; measuring a set of parameters (readouts) following exposure to the factors; and grouping genes in pathways according to similarities in such parameter measurements. The invention also provides computer-assisted analytical methods useful in said methods.

In another embodiment, the interaction between two signaling pathways, and the common component of interaction is determined by exposing a set of recombinant cells, each member of which over or under-expresses a gene in one of said pathways, to a variety of biologically active factors that are activators of signaling pathways; measuring a set of parameters (readouts) following exposure to the factors; and comparing the measured responses to determine if an over or under-expressed gene in one of said pathways responds to said activators in a manner that correlates to the responses measured for one of said over or underexpressed genes in the other of said pathways, and if such a correlation exists, determining that said pathways interact and that the common component of said interaction is the gene product for which said correlation was observed.

In another embodiment, the present invention provides a method for ordering the components of a signaling pathway by determining the epistatic relationships between combinations of activators and inhibitors of said pathway; and correlating the relative order of action of said activators and inhibitors with the order of the components of the pathway.

In yet another embodiment, the mechanism of action for a test compound is determined by exposing a set of recombinant cells, each member of which over or under-expresses a target gene, to a test compound; measuring a set of parameters in said cells following exposure to the test compound; comparing these parameter values with parameter values measured under similar conditions with a set of control compounds having known mechanisms of action; and determining that said test compound has a mechanism of action similar or identical to one of said control compounds that produces comparable parameter values under said test conditions. When required to reveal or enhance activity of the over-expressed gene, the exposing step is conducted under conditions that stimulate a signaling pathway that is the same or different from the pathway of an over-expressed gene.

A mechanism of action for a tested compound may also be determined by exposing a set of cells to an agent that specifically inhibits expression of a gene of interest, e.g. anti-sense RNA, siRNA, and the like; measuring a set of parameters in said cells following exposure to the agent; comparing these parameter values with parameter values measured under similar conditions with a tested compound; and determining that said agent has a mechanism of action similar or identical to one of said tested compound that produces comparable parameter values under said test conditions. If the profiles match, then the under-expressed gene product is the target for the compound; or the under-expressed gene product is a part of a signaling pathway and is located in the pathway near the compound target (most often just upstream or downstream); or the under-expressed gene product is a part of a protein complex, where one member of such a protein complex is targeted by the tested compound, and the other member is under-expressed gene product and disruption of any component of such a protein complex (either by compound or gene knockdown) results in a similar phenotype (functional profile).

### BRIEF DESCRIPTION OF THE DRAWINGS

FIG. 1 is a table and bar graph showing the results (SD is standard deviation) of an ELISA assay measuring ICAM-1 expression in a control (None) and six HUVEC cell lines over-expressing either TNF-alpha, IFN-gamma, IKBKB, RELA, GADD45G, or GATA3. Each of the over-expressed genes, which together represent multiple signaling pathways, resulted in a 3 to 16-fold induction of ICAM-1 expression (see Example 1.A.). These results demonstrate that measurement of a single signaling pathway response does not enable one to group gene products into a common pathway or order components in a pathway.

FIG. 2 is a table and bar graph showing the average ELISA values measured in assays for ICAM-1, VCAM-1, E-selectin, MIG, IL-8, HLA-DR, and MCP-1 using the cell lines described in regard to FIG. 1 (Example 1.B.). The results demonstrate that the response to gene over-expression of each of the additional genes or readouts is unique and distinct from the response observed for ICAM-1.

FIG. 3, part a, shows gene over-expression effects as mean log parameter expression ratios for eight parameters (CD31, E-selectin, HLA-DR, ICAM-1, IL-8, MCP-1, MIG and VCAM-1) for the genes listed in Table 2, in HUVEC incubated with IL-1-beta, with TNF-alpha, with INF-gamma, or with media alone. Shading indicates change in parameter levels: dark grey, higher level (up-regulated) compared to control; grey, no change compared to control; white, lower level (down-regulated) compared to control. Part b shows pairwise Pearson correlation coefficient calculated with mean log expression ratios using 28 parameters across IL-1-beta, TNF-alpha, INF-gamma and media alone systems combined (encompassing E-selectin, HLA-DR, ICAM-1, IL-8, MCP-1, MIG and VCAM-1 readouts from each of the four cell systems). The highest functional correlations are between genes whose products carry out the same function (e.g. MEK1* and MEK2*) or genes that are members of a common signaling pathway. Shading indicates correlations that pass statistical significance tests described further in the text and in Example 1B. Dark grey are correlation coefficients in the range of 0.75 to 1, and light grey in the range of 0.55 to 0.75. Part c shows the results of an evaluation of the similarity of functional profiles within the individual systems tested; the observed MYD88 and RAS* correlations reveal surprising system dependence. In systems lacking cytokine stimulators of NFκB, MYD88 over-expression results in up-regulation of several parameters, showing functional homology to the NFκB pathway member TNFRSF1A (TNF-receptor type I), but in the system containing IL-1-beta, in which the NFκB pathway is already strongly stimulated (and may mask any MYD88 contribution in this regard), MYD88 reveals its surprising functional similarity to RAS* to suppress IL-1-beta-induced readouts E-selectin and VCAM-1. Numbers within arrow shapes are Pearson correlation coefficients for individual systems. An * indicates constitutively active genes, with the exception of SHP2 which is dominant negative; and may stimulate NFκB by suppressing RAS/MAPK pathway.

FIG. 4. Two-dimensional representations of the relationships between over-expressed genes revealed by pairwise correlation analysis of functional profiles as described in Example 2A and FIG. 3. Twenty-eight readouts across IL-1-beta, TNF-alpha, INF-gamma and media alone systems (encompassing E-selectin, HLA-DR, ICAM-1, IL-8, MCP-1, MIG and VCAM-1 readouts from each of the four cell systems) were used for Pearson correlation analysis (shown in FIG. 3, part b). The resulting correlation matrix is presented here as a two-dimensional map where the arrangement of genes is automatically determined by multidimensional scaling, and statistically significant correlations (as determined by permutation technique, see Example 1B) are shown by the connecting lines. Only genes whose functional profiles show significant similarity to other genes are shown. Note that members of NFkB, RAS/MAPK, PI3K/Akt and IFN-γ signaling pathways fall into their respective signaling pathway clusters, while MYD88 and IRAK1 genes link to both NFκB and RAS/MAPK clusters indicating these gene products are involved in mediating interaction between the NFκB and RAS/MAPK pathways.

FIG. 5 is a table and bar graph showing the effect of NDGA on HUVEC cell lines over-expressing one of three components, TNF-alpha (TNFA in Table 2), IKBKB, and RELA, of the NFkB signaling pathway and to a control cell line, on VCAM-1 expression as measured by ELISA (see Example 3). The results demonstrate that NDGA inhibits TNF-alpha induced VCAM-1 expression, but not IKBKB or RELA induced VCAM-1 expression and prove that TNF-alpha is upstream in the pathway from IKBKB and RELA.

FIG. 6 shows a panel of drugs tested (see Example 3) and the effect of each on VCAM-1 expression (as measured by ELISA) in the HUVEC cell lines over-expressing one of the three pathway component genes TNF-alpha, IKBKB, and RELA in both a table and a linear plot (the number on the x axis corresponds to the drug number in the table). Among all the drugs tested, three compounds can inhibit either of the three test genes TNF-alpha, IKBKB, or RELA. These compounds are NDGA, ibuprofen, and SP600125. NDGA inhibits only the TNF-alpha gene, ibuprofen inhibits TNF-alpha and IKBKB genes, and SP600125 inhibits all three (TNF-alpha, IKBKB and RELA) genes.

FIG. 7 shows that drugs targeting common molecular targets induce similar system responses in gene over-expressing cells: identification of molecular targets (see Example 4). Endothelial cells expressing 16 individual genes from NFκB, RAS, PI3K/AKT and JAK/STAT (IFN-γ and IL-4) pathways were treated with compounds for 24 hours. Where indicated additional cytokines were added to cells to reveal activity of the over-expressed genes (e.g. AKT1/IL1 means that IL-1-beta was added to AKT-over-expressing cells). Parameters measured were VCAM-1 for NFκB, PI3K, and RAS/MAPK pathway genes, HLA-DR for JAK/STAT(IFNγ) pathway genes, and VCAM-1 (IL4/VCAM-1) and Eotaxin-3 (IL4/Eot3) for JAK/STAT(IL4) pathway. Part a shows a result of a pairwise Pearson correlation analysis using combined data from 20 drug-treated gene-over-expressing cells (see abscissa in part b for the list of gene-over-expressing cells). Statistically significant correlations (permutation method described further in the text and in Example 1B) in the table are shaded (dark grey for correlation coefficients in the range of 0.75 to 1, and light grey for the range of 0.55 to 0.75). Part b shows mean log expression ratios [mean values for drug/mean values for media control] of parameter (VCAM-1, HLA-DR or eotaxin-3 as described above) in cells over-expressing signaling pathway genes (see abscissa) treated with 17-AAG (5 micromolar), beta-zearelanol (5 micromolar), DRB (10 micromolar) and Apigenin (6 micromolar).

FIG. 8, part a shows effects of siRNA-mediated gene knock-down of signal activator and transducer 1 (STAT1), IFN-gamma receptor 2 (IFNGR2), Janus Kinase 1 (JAK1) or dual-knock down of extracellular signal-regulated kinases 2 and 1 (MAPK1&3 aka ERK2 and ERK1,) on expression of measured readout parameters (CD31, E-selectin, HLA-DR, ICAM-1, IL-8, MCP-1, MIG and VCAM-1) under four stimulation conditions (IL-1-beta, TNF-alpha, IFN-gamma, and IL-1beta+TNF-alpha+INF-gamma). Part b shows pairwise Pearson correlation calculated with mean log expression ratios using a string of 32 parameters (eight readouts across four systems). Statistically significant correlations (permutation method) in the table are shaded (dark grey for correlation coefficients in the range of 0.75 to 1, and light grey in the range of 0.55 to 0.75).

FIG. 9 shows two-dimensional presentation of the pairwise correlation matrix between functional profiles generated by treatment of cells with compounds, biologics or by siRNA-mediated gene knock-down. The cells used to generate functional profiles were HUVEC stimulated with a mixture of cytokines IL-1beta+TNF-alpha+IFN-gamma, and the readout parameters were E-selectin, HLA-DR, ICAM-1, IL-8, MCP-1, MIG and VCAM-1. Statistical analysis (permutation method) and generation of a two-dimensional map was done as described above. The inset shows overlapping functional profiles of siRNA (two repeat experiments) targeting TNFR gene (aka TNF-alpha receptor type I, TNFRS1A) and an antibody against TNF-alpha, a TNFR ligand (three repeat experiments).

### DETAILED DESCRIPTION OF THE INVENTION

The methods and compositions of the invention provide a system for the assessment of relationships between the components of signaling pathways, including identifying and characterizing components of a pathway; determining interactions between pathways; ordering components in a pathway; and determining the mechanism of action of a compound on a pathway. These methods enable the identification of drug targets and the corresponding mechanisms of drug action. In these methods, the consequences, or biological responses, that result from activation and inhibition at various steps along signaling pathways are measured and used to determine whether genes are in a common signaling pathway or at an intersection of two different signaling pathways; the order of action of the various components of the pathways; and the mechanism of action of a compound that affects a signaling pathway.

As used herein, the term “pathway”, or “signaling pathway” refers to a cellular interaction between two, three, four or more components, where at least one or more of the components is encoded by a gene of interest; and wherein the result of the cellular interaction is a measurable change in a biological parameter. The interaction between components may comprise physical relationships, e.g. the formation of multiprotein complexes; and/or functional relationships; e.g. phosphorylation, translocation etc. of a component. The physical and functional aspects may be combined, e.g. the formation of a stable complex that results in activation of a component. Frequently, there is a cascade of activation or inhibition of components in a pathway, which result in a physiological change, e.g. in gene expression; synthesis of metabolites; voltage potential across a cell membrane; release of neurotransmitters; changes in intracellular concentrations of ions; and the like.

Signaling pathways are composed of multi-protein complexes (e.g. receptor with its receptor-associated factors) and components that may shuttle between such complexes (e.g. NFκB transcription factor shuttles between a kinase and a proteasome complexes in cytoplasm and a transcriptional complexes in the nucleus). Affecting any of the individual components of a signaling pathway, either those that are part of a multi-protein complex or those that are independent, may result in a similar functional outcome, and thus will be useful for practicing methods for signaling pathway mapping.

In most instances, a signaling pathway will comprise a signal transduction component, where there is a conversion of a signal from one form to another, e.g. the binding of a factor to a cell surface receptor may be transduced into an alteration of cellular levels of Ca⁺⁺ or cAMP.

Signaling pathways are frequently complex, and the methods of the invention may be utilized in determining relationships between components in a subset of a pathway or pathways, and need not involve all of the components. The elements of a number of pathways have been described in the art, however it is often uncertain how different pathways interact, or how and where a new component fits into a pathway. The methods of the invention provide a means to obtain this information.

Datasets of information are obtained from biologically multiplexed activity profiling (BioMAP®) of cells; usually cells that have been genetically modified to over or under-express a gene of interest. Such methods are described, for example, in U.S. Pat. No. 6,656,695; in co-pending U.S. provisional patent application 60/465,152, filed Apr. 23, 2003; in co-pending U.S. provisional patent application 60/539,447, filed Jan. 26, 2004; and U.S. patent applications U.S. Ser. No. 09/952,744, filed Sep. 13, 2001; U.S. Ser. No. 10/220,999; and U.S. Ser. No. 10/236,558, filed Sep. 5, 2002, herein each specifically incorporated by reference. As used herein, the term “a set” of cells refers to at least two, at least three, at least four, or more distinct cell types, where the cells may differ by derivation, e.g. endothelial cells, including primary endothelial cells; peripheral blood mononuclear cells; smooth muscle cells; cancer cells; neural cells; etc. The cells may also differ in the modified gene, e.g. a set of cells may comprise endothelial cells modified to over or under express components of the TGF-β signaling pathway, e.g. TGF-β receptor type I, TGF-βreceptor type II; TAB-1; TAK-1; MAPKK; MAPK; Smad2; Smad4; and TGF-β.

Briefly, the methods provide screening assays where the effect of altering cells in culture is assessed by monitoring multiple output parameters. The result is a dataset that can be analyzed for the effect of a genetic agent on a signaling pathway, for determining the pathways in which an agent acts, for grouping agents that act in a common pathway, for identifying interactions between pathways, and for ordering components of pathways.

Screening methods of interest utilize a systems approach to characterization of signaling pathways based on statistical analysis of parameter data sets from human cell-based systems. In these models, biological complexity may be provided by the activation of multiple signaling pathways; interactions of multiple human cell types; and/or the use of multiple systems for data analysis. These model systems are surprisingly robust, reproducible, and responsive to and discriminatory of the activities of a large number of genetic agents.

In the methods of the invention, the analysis of the function of signaling pathways in cells is carried out by measuring individual parameters and combinations of parameters under multiple parallel cell stimulation conditions. These parameters reflect the operation of signaling pathways and so can include cellular products, epitopes, or functional states, whose levels vary in abundance or activity in response to activators or inhibitors of the signaling pathways.

For example, a set of recombinant cells, each member of which over or under-expresses a gene to be identified either as a gene in the pathway or not in the pathway, may be exposed to a variety of biologically active factors that are either activators or inhibitors of signaling pathways. Alternatively, a set of recombinant cells, each member of which over or under-expresses a gene a first pathway that is being analyzed with respect to a second pathway is exposed to a variety of biologically active factors that are activators of signaling pathways and compared to determine if an over or under-expressed gene in one of said pathways responds to the activators in a manner that correlates to the responses measured for one of said over or under-expressed genes in the other of said pathways. A set of cells may be exposed to a variety of biologically active factors that are activators of signaling pathways, and the results correlated for the relative order of action of the activators and inhibitors. A set of recombinant cells may be exposed to a test compound under test conditions and compared to the results of exposure with a known compound. A mechanism of action for a tested compound may also be determined by exposing a set of cells to an agent that specifically inhibits expression of a gene of interest, and comparing the results obtained with the specific inhibitor to the results obtained with the tested compound.

For the purpose of present invention, activators are defined as molecules, drugs, genetic modifications, functional states, or conditions that activate or stimulate signaling pathways. Naturally occurring molecules or conditions usually activate the signaling machinery from an upstream position in a pathway and so generally reflect naturally occurring biological processes. Other activators, such as pharmaceutical drugs, may function at sites internal to a pathway and so act in a manner that does not usually occur during normal cellular function. In all cases, activators get the signal moving, or keep it moving, along the signaling pathway. Activators initiate signaling, stimulate or activate a pathway, turn-on a pathway, or keep a signaling pathway turned on.

Activators useful in the practice of the present invention include, but are not limited to, biological materials of natural or recombinant origin, including cytokines, growth factors, interleukins, hormones, peptides, proteins, DNAs, RNAs, carbohydrates, and lipids. Activators useful in the practice of the present invention also include synthetic or naturally occurring compounds, such as small, medium or large organic molecules, drugs, and inorganic molecules. Activators useful in the practice of the present invention also include environmental conditions, such as temperature, pH, humidity, light, pressure, co-culture with cells of a different type, and irradiation with UV, gamma, x-rays, or particle beams. Activators useful in the practice of the present invention also include conditions resulting from the genetic or other modification of cells, such as gene over-expression, gene deletion, functional gene knock-out or knock-in, expression of constitutively active components, expression of dominant negative components, expression of anti-sense RNA, siRNA, and expression of mutant components with altered activity, such as, for example, expression of components which are defective, partially defective or hypersensitive.

In some cases, it may be advantageous or necessary to combine two or more factors to activate a pathway of interest. For example, co-culture of cells with cells of a different type in combination with the application of cytokines, or a mixture of cytokines and growth factors, may be required to activate a particular pathway.

Inhibitors useful in the practice of the present invention include molecules, drugs, genetic modifications, functional states, or conditions that inhibit signaling pathways. Inhibitors block the signal from moving along a signaling pathway. Illustrative inhibitors include drugs that act to block, obstruct, or impede the transmission of the signal along the pathway, typically by interacting with one of the components of the pathway and rendering that component functionally inactive.

Inhibitors useful in the methods of the present invention include the same types of materials and conditions discussed above with respect to activators, differing only with respect to their effect on the pathway of interest. Thus, inhibitors useful in the methods of the invention include, but are not limited to, biological compounds of natural or recombinant origin and other compounds of natural or synthetic origin, such as drugs, small, medium, or large organic molecules, cytokines, growth factors, interleukins, hormones, peptides, proteins, DNAs, RNAs, carbohydrates, lipids, and inorganic molecules. Likewise, inhibitors useful in the methods of the invention include environmental conditions, such as temperature, pH, humidity, light, pressure, co-culture with cells of a different type, and irradiation with UV, gamma, x-rays, or particle beams. Likewise, inhibitors include conditions resulting from the genetic modification of cells, such as gene over-expression, gene deletion, functional gene knock-out or knock-in, expression of constitutively active components, expression of dominant negative components, expression of anti-sense RNA, siRNA, and expression of mutant components with altered activity, for example expression of components that are defective, partially defective or hypersensitive. As is the case with activators, in some instances, a combination of two or more factors may be useful or required to inhibit a particular pathway.

Activators and inhibitors are distinguished by the different effects each has on the function of signaling pathways as determined by measuring specific individual parameters and combinations of parameters. Thus, activators and inhibitors are not distinguished by the types of molecules or the methods by which modification of signaling is achieved. Both activators and inhibitors cause perturbations, modifications, or alterations to signaling pathways. A variety of activators and inhibitors (which may also be referred to as “factors”) of signaling pathways have been, and continue to be, identified. The methods of the invention can be practiced with a wide variety of activators and inhibitors, including those not yet identified in the scientific literature.

The application of an activator or inhibitor does not necessarily result in measurable phenotypic responses, i.e. alterations in parameter levels, in normal cells. In some cases, the action of an activator or inhibitor may only be observed when particular conditions are met. For example, a gene that inhibits a particular step in a signaling pathway may have little or no effect when applied to cells that have an inactive signaling pathway. The effect of signaling by a gene may only become evident when the pathway is active or stimulated. Thus activators and inhibitors may reveal their activities only under specific conditions.

In the typical situation, an activator will activate or turn on a signaling pathway, which results in transmission of a signal down the pathway and causes the measured level of one or more parameters to vary. In the typical case, application of an inhibitor to an activated pathway will block transmission of the signal at some point along the pathway, and the measured level of one or more parameters will return to the level observed before the pathway was activated.

As used herein, the term “genetic agent” refers to polynucleotides and analogs thereof, which are used in the methods of the invention to genetically alter cells such that the cell over or under expresses a gene of interest. Genetic agents such as DNA can result in an experimentally introduced change in the genome of a cell, generally through the integration of the sequence into a chromosome. Genetic changes can also be transient, where the exogenous sequence is not integrated but is maintained as an episomal agent. Genetic agents, such as siRNA, or antisense oligonucleotides, can also affect the expression of proteins without changing the cell's genotype, by interfering with the transcription or translation of mRNA. The effect of a genetic agent is to increase or decrease expression of one or more gene products in the cell.

Introduction of an expression vector encoding a polypeptide can be used to express the encoded product in cells lacking the sequence, or to over-express the product. Various promoters can be used that are constitutive or subject to external regulation, where in the latter situation, one can turn on or off the transcription of a gene. These coding sequences may include full-length cDNA or genomic clones, fragments derived therefrom, or chimeras that combine a naturally occurring sequence with functional or structural domains of other coding sequences. Alternatively, the introduced sequence may encode an anti-sense sequence; be an anti-sense oligonucleotide; encode a dominant negative mutation, or dominant or constitutively active mutations of native sequences; altered regulatory sequences, etc.

In addition to sequences derived from the host cell species, other sequences of interest include, for example, genetic sequences of pathogens, for example coding regions of viral, bacterial and protozoan genes, particularly where the genes affect the function of human or other host cells. Sequences from other species may also be introduced, where there may or may not be a corresponding homologous sequence.

A large number of public resources are available as a source of genetic sequences, e.g. for human, other mammalian, and human pathogen sequences. A substantial portion of the human genome is sequenced, and can be accessed through public databases such as Genbank. Resources include the Uni-gene set, as well as genomic sequences. For example, see Dunham et al. (1999) Nature 402, 489-495; or Deloukas et al. (1998) Science 282, 744-746.

cDNA clones corresponding to many human gene sequences are available from the IMAGE consortium. The international IMAGE Consortium laboratories develop and array cDNA clones for worldwide use. The clones are commercially available, for example from Invitrogen Corporation, Carlsbad, Calif. Methods for cloning sequences by PCR based on DNA sequence information are also known in the art.

In one embodiment, the genetic agent is an antisense sequence that acts to reduce expression of the complementary sequence. Antisense nucleic acids are designed to specifically bind to RNA, resulting in the formation of RNA-DNA or RNA-RNA hybrids, with an arrest of DNA replication, reverse transcription or messenger RNA translation. Antisense molecules inhibit gene expression through various mechanisms, e.g. by reducing the amount of mRNA available for translation, through activation of RNAse H, or steric hindrance. Antisense nucleic acids based on a selected nucleic acid sequence can interfere with expression of the corresponding gene. Antisense nucleic acids can be generated within the cell by transcription from antisense constructs that contain the antisense strand as the transcribed strand.

The anti-sense reagent can also be antisense oligonucleotides (ODN), particularly synthetic ODN having chemical modifications from native nucleic acids, or nucleic acid constructs that express such anti-sense molecules as RNA. One or a combination of antisense molecules may be administered, where a combination may comprise multiple different sequences. Antisense oligonucleotides will generally be at least about 7, usually at least about 12, more usually at least about 20 nucleotides in length, and not more than about 500, usually not more than about 50, more usually not more than about 35 nucleotides in length, where the length is governed by efficiency of inhibition, specificity, including absence of cross-reactivity, and the like.

A specific region or regions of the endogenous sense strand mRNA sequence is chosen to be complemented by the antisense sequence. Selection of a specific sequence for the oligonucleotide may use an empirical method, where several candidate sequences are assayed for inhibition of expression of the target gene. A combination of sequences may also be used, where several regions of the mRNA sequence are selected for antisense complementation.

Alternatively, RNAi technology is an effective approach for inhibiting expression of a target gene by a process in which double-stranded RNA is introduced into cells expressing a candidate gene to inhibit expression of the candidate gene, i.e., to “silence” its expression. The dsRNA is selected to have substantial identity with the candidate gene. It is believed that dsRNA suppresses the expression of endogenous genes by a post-transcriptional mechanism. Specificity in inhibition is important because accumulation of dsRNA in mammalian cells can result in the global blocking of protein synthesis. The dsRNA is prepared to be substantially identical to at least a segment of a target gene. Suitable regions of the gene include the 5′ untranslated region, the 3′ untranslated region, and the coding sequence. The dsRNA may consist of two separate complementary RNA strands or a single strand of RNA that is self-complementary, such that the strand loops back upon itself to form a hairpin loop. Regardless of form, RNA duplex formation can occur inside or outside of a cell. Generally, the dsRNA is at least 10-15 nucleotides long. dsRNA can be prepared according to any of a number of methods that are known in the art, including in vitro and in vivo methods, as well as by synthetic chemistry approaches.

As an alternative method, dominant negative mutations are readily generated for corresponding proteins. These may act by several different mechanisms, including mutations in a substrate-binding domain; mutations in a catalytic domain; mutations in a protein binding domain (e.g. multimer forming, effector, or activating protein binding domains); mutations in cellular localization domain, etc. See Rodriguez-Frade et al. (1999) P.N.A.S. 96:3628-3633; suggesting that a specific mutation in the DRY sequence of chemokine receptors can produce a dominant negative G protein linked receptor; and Mochly-Rosen (1995) Science 268:247.

Methods that are well known to those skilled in the art can be used to construct expression vectors containing coding sequences and appropriate transcriptional and translational control signals for increased expression of an exogenous gene introduced into a cell. These methods include, for example, in vitro recombinant DNA techniques, synthetic techniques, and in vivo genetic recombination. Alternatively, RNA capable of encoding gene product sequences may be chemically synthesized using, for example, synthesizers. See, for example, the techniques described in “Oligonucleotide Synthesis”, 1984, Gait, M. J. ed., IRL Press, Oxford.

A variety of host-expression vector systems may be utilized to express a genetic coding sequence. Expression constructs may contain promoters derived from the genome of mammalian cells, e.g., metallothionein promoter, elongation factor promoter, actin promoter, etc., from mammalian viruses, e.g., the adenovirus late promoter; the vaccinia virus 7.5K promoter, SV40 late promoter, cytomegalovirus, etc. In mammalian host cells, a number of viral-based expression systems may be utilized, e.g. retrovirus, lentivirus, adenovirus, herpesvirus, and the like.

In a preferred embodiment, methods are used that achieve a high efficiency of transfection, and therefore circumvent the need for using selectable markers. These may include adenovirus infection (see; for example Wrighton, 1996, J. Exp. Med. 183: 1013; Soares, J. Immunol., 1998, 161: 4572; Spiecker, 2000, J. Immunol 164: 3316; and Weber, 1999, Blood 93: 3685); and lentivirus infection (for example, International Patent Application WO000600; or WO9851810). Adenovirus-mediated gene transduction of endothelial cells has been reported with 100% efficiency. Retroviral vectors also can have a high efficiency of infection with endothelial cells, provides virtually 100% report a 40-77% efficiency. Other vectors of interest include lentiviral vectors, for examples, see Barry et al. (2000) Hum Gene Ther 11(2):323-32; and Wang et al. (2000) Gene Ther 7(3):196-200.

The methods of the present invention enable one with no prior knowledge about a signaling pathway to identify the components of the pathway, identify the components in the pathway that interact with other signaling pathways, order the components of the pathway, and identify the mechanism of action of a compound by identification of the component of a signaling pathway that is the target of action of the compound. Each of these methods is discussed below and exemplified in the following examples. In the absence of knowledge about one or more components of a signaling pathway, the methods of the invention can be practiced using gene over- or under-expression (optionally plus activation and/or inhibition) to cluster genes into one or more signaling pathways.

In this method, measurement of the pathway response to gene over- or under-expression under a set of test conditions is used to cluster genes into functional groups. Those genes that induce highly similar responses in cells, preferably across multiple different test condition, are identified as belonging to a signaling pathway. This methodology is illustrated in Examples 1.B. and 2.A, below, while Example 1.A. below demonstrates that measurement of a single parameter is insufficient for such clustering.

The present invention also provides methods for determining if two or more signaling pathways interact, and if such interaction exists, then the point in the pathway where such an intersection occurs. These methods utilize the analysis of a number of potential pathway components under a number of stimulatory and/or inhibitory conditions using a set of cells that over- or under-express at least one of the pathway components of interest. The pathway-specific responses to these conditions in these sets of cells are compared and analyzed to determine if there are correlations. Such correlations can be used to predict not only that certain components are in the same pathway (as illustrated in Examples 1.B. and 2.A, below) but also that components are in two different pathways that interact and the point of interaction. This aspect of the invention is illustrated in Examples 2.B. and 2C, below.

The invention also provides methods that enable one to arrange the genes of a signaling pathway in the order by which a signal is transferred from one member of the signaling pathway to the other. In these methods, a set of cells, each member of which over-expresses a gene in the pathway to be ordered (and so has been activated with respect to that over-expressed gene product and the pathway(s) in which it is involved), is exposed to active concentrations of a set of inhibitors of gene function. A number of parameters indicative of pathway activity are measured, and the measurements used to determine the order of genes in the pathway. This method of the invention is illustrated in Example 3, below.

This pathway ordering method of the invention thus involves the identification of the relative order of action of a set of activators and inhibitors for a signaling pathway through the systematic determination of the epistatic relationships between all possible combinations of a set of activator-inhibitor pairs. These relationships, in combination with other available information about the activators and inhibitors, provide a framework for pathway architecture. In one embodiment, the method is practiced by conducting a systematic combination of tests using two or more activators and two or more inhibitors to determine relationships between the components of signaling pathways. By providing the order of the components of a pathway—the order in which the signal moves through the pathway—the pathway ordering method enables the identification of drug targets and the corresponding mechanisms of drug action.

The activators and inhibitors employed in the pathway ordering method influence the measured level of at least one parameter in common. If the activators and inhibitors influence the same parameter, or a combination of parameters, then one can infer that those activators and inhibitors are affecting the same signaling pathway. This inference can be strengthened by increasing the number of parameters measured and identifying additional parameters that vary in a similar way. Thus, the higher the correlation between the profiles of measured parameter variations for a given set of activators and inhibitors, the more preferred those activators and inhibitors are for purposes of the present invention.

This pathway ordering method of the present invention therefore involves the measurement of the response of a signaling pathway to at least two or more activators and at least two or more inhibitors that act on that signaling pathway. The responses measured enable one to identify the relative order of action of the activators and inhibitors. This relative order of action of activators and inhibitors is then used to deduce relationships between the components of the pathway. In turn, those component relationships can be used to identify drug targets and the mechanism of drug action, based on the identities of and available information about the particular activators and inhibitors used in a particular application of the method.

The most significant and direct effects of the vast majority of activators and inhibitors of signaling pathways occur at individual steps along the pathway. Any particular activator or inhibitor generally exerts its effect on the pathway by activating or inhibiting a particular component and thereby a particular step in the pathway. It should be noted that while the methods of the invention can be practiced with “direct” inhibitors or activators, i.e., compounds that act directly on a pathway component, “indirect” inhibitors or activators can be employed as well. For example, an inhibitor can be specific for a gene or gene product in the pathway (e.g. specific chemical inhibitor, inhibitory antibody or antisense generated against a gene in the pathway) and so be a “direct inhibitor”, but another inhibitor, an “indirect inhibitor” can act on a gene product that is part of a different pathway than the pathway of interest but inhibition of which results in the inhibition of the pathway of interest. Many signaling pathways in cells are interconnected and co-dependent, and if the point of interaction of two signaling pathways is downstream of the point of activation of the activator (for example, an over-expressed gene product), then such “indirect inhibitor” will have an inhibitory effect and can be used in the method.

The pathway ordering method of the invention involves the determination of the relative order of action of a set of activators and inhibitors for a signaling pathway by examining the effects of the combined application of all possible activator-inhibitor pairs from all of the inhibitors and activators examined. If an inhibitor blocks pathway stimulation by an activator, then the inhibitor is acting downstream from the point of action of the activator. If an inhibitor does not block pathway stimulation by an activator, then the inhibitor is acting upstream from the point of action of the activator. If an activator and an inhibitor both act on the same component of the pathway, then, the relative strengths of activation versus inhibition will determine the apparent upstream-downstream relationship, and a dose-response analysis can be used to determine that the point of action is identical.

By combining the upstream-downstream (epistatic) relationships between all of the activator-inhibitor pairs, a map of the pathway is constructed. This map can be enhanced by the addition of any available information concerning the identity of the activators and inhibitors employed in the analysis. For example, activators may have been generated by the over-expression of genes for identified components of the pathway; thus, such activators correspond to known pathway components. Practice of the invention leads to a better understanding of signaling pathway architecture and drug-target interactions.

With the above methods one can identify the components of a signaling pathway as well as the components that define the points of interaction between two pathways and to order the components in a pathway. Indirect inhibitors for which molecular target is known are especially useful in this regard. When an indirect inhibitor is used to determine the order of genes in a signaling pathway, a point between an upstream gene that is sensitive to inhibition by the indirect inhibitor and the downstream gene that is not affected by the indirect inhibitor is the point of interaction of the studied signaling pathway and the pathway to which the molecular target for the indirect inhibitor belongs. This information provides the basis for powerful new methods provided by the invention to determine the mechanism of action of a drug, as illustrated in Example 4, below. In these methods, the test compound is contacted with a set of cells comprising members that over-express a gene of interest that may be a target of the compound. In some embodiments of the method, the set of cells can represent all of the genes in a single pathway or in multiple pathways. A set of parameters is measured in the cells contacted with the compound, and the measured parameters are compared with the measurements taken for control compounds, with known mechanisms of action, to determine which control compound produces parameter measurements most similar to those measured for the test compound. The mechanism of action of the test compound is thereby determined to be that of the control compound to which it is most identical.

For those compounds for which the mechanism of action is unique—previously not observed or known to be a property of any known compound, the other methods of the invention can be used to define a specific mechanism of action. Thus, the compound can be used as a factor in the gene clustering/pathway identification method to identify the pathway(s) it affects, and then used with other known activators or inhibitors of that pathway in the pathway ordering method of the invention to identify the precise point of action on the pathway. In the event that no other known compound is known to affect the pathway affected by the test compound, then the mechanism of action determining method of the invention can be used as a screen to identify other compounds that behave similarly to the test compound. Then, these other compounds are used with the test compound in the pathway ordering method of the invention to identify the precise point of action on the pathway.

Gene specific inhibitors, e.g. RNAi, ribozymes, antisense RNA, antisense oligonucleotides, intracellular antibodies, etc. can be used in place of chemical inhibitors for creating activator-inhibitor pairs required for pathway ordering. Furthermore, functional profiles generated using those specific inhibitors can be compared to functional profiles obtained with chemical compounds of unknown function, and if the profiles match, one can conclude that they share the same molecular target, or distinct molecular targets but which are a part of the same protein complex, where inhibiting any of the components of a complex would result in a similar functional profile.

Thus, the present invention provides a number of related and complementary methods that can be used in a wide variety of applications and combinations in drug discovery and development. The methods of the invention find application not only in screening compounds to identify drug development candidates and compounds that serve as starting points for making analogs to determine structure-activity relationships and make compounds with improved properties but also to characterize drugs already in pre-clinical or clinical development or even marketed drugs to identify those with potential side-effect problems (due to the drug having off-target activity, as can be identified using the mechanism of action determination method of the invention) or lack thereof.

The data from a typical “system”, as used herein, provides a single cell type or combination of cell types (where there are multiple cells present in a well) in an in vitro culture condition. Primary cells are preferred, or cells derived from primary cells. In a system, the culture conditions provide a common biologically relevant context. Each system comprises a control, e.g. the cells in the absence of the genetic agent or test compound, although often in the presence of the factors in the biological context. The samples in a system are usually provided in triplicate, and may comprise one, two, three or more triplicate sets.

As used herein, the biological context refers to the environment, including exogenous factors added to the culture, which factors stimulate pathways in the cells. Numerous factors are known that induce pathways in responsive cells. By using a combination of factors to provoke a cellular response, one can investigate multiple individual cellular physiological pathways and simulate the physiological response to a change in environment.

A BioMAP® dataset comprises values obtained by measuring parameters or markers of the cells in a system. Each dataset will therefore comprise parameter output from a defined cell type(s) and biological context, and will include a system control. As described above, each sample, e.g. candidate agent, genetic construct, etc., will generally have triplicate data points; and may be multiple triplicate sets. Datasets from multiple systems may be concatenated to enhance sensitivity, as relationships in pathways are strongly context-dependent. It is found that concatenating multiple datasets by simultaneous analysis of 2, 3, 4 or more systems will provide for enhance sensitivity of the analysis.

By referring to a BioMAP® is intended that the dataset will comprise values of the levels of at least two sets of parameters, preferably at least three parameters, more preferably 4 parameters, and may comprise five, six or more parameters.

The parameters may be optimized by obtaining a system dataset, and using pattern recognition algorithms and statistical analyses to compare and contrast different parameter sets. Parameters are selected that provide a dataset that discriminates between changes in the environment of the cell culture known to have different modes of action, i.e. the biomap (functional profile) is similar for agents with a common mode of action, and different for agents with a different mode of action. The optimization process allows the identification and selection of a minimal set of parameters, each of which provides a robust readout, and that together provide a biomap (functional profile) that enables discrimination of different modes of action of stimuli or agents. The iterative process focuses on optimizing the assay combinations and readout parameters to maximize efficiency and the number of signaling pathways and/or functionally different cell states produced in the assay configurations that can be identified and distinguished, while at the same time minimizing the number of parameters or assay combinations required for such discrimination. Optimal parameters are robust and reproducible and selected by their regulation by individual factors and combinations of factors.

Parameters (readouts) are quantifiable components of cells. A parameter can be any cell component or cell product including cell surface determinant, receptor, protein or conformational or posttranslational modification thereof, lipid, carbohydrate, organic or inorganic molecule, nucleic acid, e.g. mRNA, DNA, etc. or a portion derived from such a cell component or combinations thereof. While most parameters will provide a quantitative readout, in some instances a semi-quantitative or qualitative result will be acceptable. Readouts may include a single determined value, or may include mean, median value or the variance, etc.

Selection of parameters is based on the following criteria, where any parameter need not have all of the criteria: the parameter is modulated in the physiological condition that one is simulating with the assay combination; the parameter has a robust response that can be easily detected and differentiated; the parameter is not co-regulated with another parameter, so as to be redundant in the information provided; and in some instances, changes in the parameter are indicative of toxicity leading to cell death. The set of parameters selected is sufficiently large to allow distinction between datasets, while sufficiently selective to fulfill computational requirements.

Parameters of interest include detection of cytoplasmic, cell surface or secreted biomolecules, frequently biopolymers, e.g. polypeptides, polysaccharides, polynucleotides, lipids, etc. Cell surface and secreted molecules are a preferred parameter type as these mediate cell communication and cell effector responses and can be readily assayed. In one embodiment, parameters include specific epitopes. Epitopes are frequently identified using specific monoclonal antibodies or receptor probes. In some cases the molecular entities comprising the epitope are from two or more substances and comprise a defined structure; examples include combinatorially determined epitopes associated with heterodimeric integrins. A parameter may be detection of a specifically modified protein or oligosaccharide, e.g. a phosphorylated protein, such as a STAT1 transcription factor; or sulfated oligosaccharide, or such as the carbohydrate structure Sialyl Lewis x, a selectin ligand. The presence of the active conformation of a receptor may comprise one parameter while an inactive conformation of a receptor may comprise another, e.g. the active and inactive forms of heterodimeric integrin α_{M}β₂ or Mac-1.

Where a test compound is used, the compound may be drawn from numerous chemical classes, primarily organic molecules, which may include organometallic molecules, inorganic molecules, genetic sequences, etc. An important aspect of the invention is to evaluate candidate drugs, select therapeutic antibodies and protein-based therapeutics, with preferred biological response functions. Candidate agents comprise functional groups necessary for structural interaction with proteins, particularly hydrogen bonding, and typically include at least an amine, carbonyl, hydroxyl or carboxyl group, frequently at least two of the functional chemical groups. The candidate agents often comprise cyclical carbon or heterocyclic structures and/or aromatic or polyaromatic structures substituted with one or more of the above functional groups. Candidate agents are also found among biomolecules, including peptides, polynucleotides, saccharides, fatty acids, steroids, purines, pyrimidines, derivatives, structural analogs or combinations thereof.

Included are pharmacologically active drugs, genetic agents, etc. Compounds of interest include chemotherapeutic agents, anti-inflammatory agents, hormones or hormone antagonists, ion channel modifiers, and neuroactive agents. Exemplary of pharmaceutical agents suitable for this invention are those described in, “The Pharmacological Basis of Therapeutics,” Goodman and Gilman, McGraw-Hill, New York, N.Y., (1996), Ninth edition, under the sections: Drugs Acting at Synaptic and Neuroeffector Junctional Sites; Drugs Acting on the Central Nervous System; Autacoids: Drug Therapy of Inflammation; Water, Salts and Ions; Drugs Affecting Renal Function and Electrolyte Metabolism; Cardiovascular Drugs; Drugs Affecting Gastrointestinal Function; Drugs Affecting Uterine Motility; Chemotherapy of Parasitic Infections; Chemotherapy of Microbial Diseases; Chemotherapy of Neoplastic Diseases; Drugs Used for Immunosuppression; Drugs Acting on Blood-Forming organs; Hormones and Hormone Antagonists; Vitamins, Dermatology; and Toxicology, all incorporated herein by reference. Also included are toxins, and biological and chemical warfare agents, for example see Somani, S. M. (Ed.), “Chemical Warfare Agents,” Academic Press, New York, 1992).

The data may be subjected to non-supervised hierarchical clustering to reveal relationships among profiles. For example, hierarchical clustering may be performed, where the Pearson correlation is employed as the clustering metric. Clustering of the correlation matrix, e.g. using multidimensional scaling, enhances the visualization of functional homology similarities and dissimilarities. Multidimensional scaling (MDS) can be applied in one, two or three dimensions. Application of MDS produces a unique ordering for the agents, based on the distance of the agent profiles on a line. To allow objective evaluation of the significance of all relationships between compound activities, profile data from all multiple systems may be concatenated; and the multi-system data compared to each other by pairwise Pearson correlation. The relationships implied by these correlations may then be visualized by using multidimensional scaling to represent them in two or three dimensions.

Biological datasets are analyzed to determine statistically significant matches between datasets, usually between test datasets and control, or profile datasets. Comparisons may be made between two or more datasets, where a typical dataset comprises readouts from multiple cellular parameters resulting from exposure of cells to biological factors in the absence or presence of a candidate agent, where the agent may be a genetic agent, e.g. expressed coding sequence; or a chemical agent, e.g. drug candidate.

A prediction envelope is generated from the repeats of the control profiles; which prediction envelope provides upper and lower limits for experimental variation in parameter values. The prediction envelope(s) may be stored in a computer database for retrieval by a user, e.g. in a comparison with a test dataset.

The raw data may be initially analyzed by measuring the values for each parameter, usually in triplicate or in multiple triplicates. For each gene or agent in a system, the mean value for each parameter is calculated; and divided by the mean parameter value from a negative control sample to generate a ratio. The ratios are then log₁₀ transformed. The transformed ratios may be averaged from repeat experiments of a system. The dataset thus obtained may be referred to as a normalized biomap dataset.

The “prediction envelope” methodology provides a non-parametric approach for establishing the significance of a profile. Methods of generating a prediction envelope may include a non-centered “prediction envelope”; centered “prediction envelope”; “centered prediction envelope” based on Hottling's T² method; and the like.

For a non-centered “prediction envelope” method, profiles that correspond to the control from many experiments are collected. These profiles contain a number of parameter values. The values that correspond to the measurement of each parameter can be the individual measurement from a well, the average of the replicates measured in the experiment, the median of the replicates, etc. Visually, a 1-standard deviation envelope may be created around the profile of the combined means by connecting the points that correspond to the values of one standard deviation for each of the measured values for the parameters.

These two “envelope” lines are then moved parallel to themselves, by equal distances, outwards until a specific number of the control profiles are completely contained within them and a user specified number has at least one of the measured parameters outside them. The prediction level of the envelope is specified as the percentage of control curves that are completely contained within the “prediction envelope”.

To create a centered “prediction envelope” requires the use of two sets of control replicates on each plate. These replicates provide a variability estimate for the combination of system and readout measurement on the given plate. Each set provides a point estimate for the parameter value. This point estimate can be obtained as the mean of the replicates, the median, etc. The overall mean of the two points is calculated and subtracted from the two point estimates thus centering the points around zero. Combining the points from all parameters of an experiment, one obtains a profile (symmetric lines around zero) representing an estimate of the control variability for the given experiment. Similar profiles from many experiments are used to create a “centered prediction envelope” using methodology identical to the one employed previously. Centered profiles of estimated variability may also be transformed into an equivalent single “distance” value. Centered profiles from multiple experiments are collected and the covariance matrix of the set is calculated. Then, forming the quadratic form of the profile vector and the covariance matrix, a single numerical value is obtained that represents the “distance” of each control profile from the “center” of all control profiles. An empirical distribution of these distances, that represent the variability of the control profile across many experiments, is obtained. This distribution provides the means of predicting the expected variability of the control in a subsequent experiment at a predefined prediction level. This methodology has the additive advantage of accounting for the possible covariance of the readouts comprising the profile.

A profile is considered to be different than the control if at least one of the parameter values of the profile exceeds the “prediction envelope” limits that correspond to a predefined level of significance. The test for significance depends on the type of “prediction envelope” that is selected. For the non-centered “prediction envelope”, the test agent profile is compared against the envelope that has been calculated at the predefined significance level.

For the centered “prediction envelope” the ratio of the test agent profile to the control profile is formed by dividing the corresponding OD values of the agent and the control parameters. This operation is equivalent to centering the test agent profile in order to make it compatible with the centered envelope created at a predefined significance level (the normalization and transformation operations should be identical for consistency). For the third method, the test agent profile is again centered by dividing with the corresponding control profile and the quadratic form of the centered profile and the covariance matrix of the controls is formed. The value obtained from this multiplication is then compared with the value obtained from the control variance distribution at the required significance level.

The data may be subjected to non-supervised hierarchical clustering to reveal relationships among profiles. For example, hierarchical clustering may be performed, where the Pearson correlation is employed as the clustering metric. Clustering of the correlation matrix, e.g. using multidimensional scaling, enhances the visualization of functional homology similarities and dissimilarities. Multidimensional scaling (MDS) can be applied in one, two or three dimensions.

Application of 1D MDS produces a unique ordering for the pathway components, based on the distance of the components on a line. The rows and columns of the original matrix are then reordered to reflect the result of MDS. In the combination of multidimensional scaling and pivoting to move high correlations toward the diagonal: for each row, in the reordered pairwise correlation matrix, starting from the first and moving towards the last, is the rank of the correlation coefficients between the diagonal element and the last element on the row. The columns (and due to symmetry the rows) are then reordered so that the rank of the correlation coefficients is decreasing from the diagonal towards the limit of the matrix. These steps are repeated until all rows are processed. Once the connectivity of the nodes is established the results may be visually displayed for enhanced information accessibility to a user. In one embodiment, the results are displayed as a network.

However, hierarchical clustering with a binary comparison method can obscure significant similarities between compounds that are on different branches of a tree. This becomes particularly problematic as the number of variables (parameters and systems) increases. To allow objective evaluation of the significance of all relationships between compound activities, profile data from all multiple systems may be concatenated; and the multi-system data compared to each other by pairwise Pearson correlation. The relationships implied by these correlations may then be visualized by using multidimensional scaling to represent them in two or three dimensions.

In order to accomplish this, multidimensional scaling is used on the original profiles, transforming each one of them into a point in 2D or 3D space. The use of MDS for this operation is preferred because it preserves the relative distance of the nodes. Distances between agents are representative of their similarities and lines are drawn between compounds whose profiles are similar at a level not due to chance.

These and other aspects of the invention will be appreciated by those of skill in the art upon contemplation of the preceding detailed description of the invention and the following Examples, which are presented solely for illustrative purposes. As those of skill in the art will appreciate, the methods of the present invention can be applied to a wide variety of pathways, pathway components, and cells using conditions, inhibitors, activators and measuring responses other than those described in the Examples below.

### EXAMPLE 1

### Grouping Genes in a Signal Transduction Pathway

This Example illustrates a method of the invention for grouping genes in a signal transduction pathway. In part A, a single response of a signal transduction pathway is analyzed under a variety of conditions simply to demonstrate that such a measurement is insufficient either to place components in a single pathway or to order components in that pathway, because different signal transduction pathway components can generate the same response when stimulated. In part B, multiple responses of signal transduction pathways are analyzed to show that, when such responses are compared, correlations can be used to deduce that various components are in the same pathway but that one cannot infer the order of such components in the pathway from those correlations.

A. Measuring a Single Response to Stimulation of a Signal Transduction Pathway. This Example 1.A. demonstrates that over-expression of several different genes can activate a signal transduction pathway, even though all of those different genes do not produce components of the pathway. In this Example 1.A, ICAM-1 expression is the single pathway response measured as a result of over-expression of genes for soluble factors TNF-alpha and IFN-gamma, IkB kinase beta (IKBKB), transcription factors RELA and GATA3, and stress-response gene GADD45G. TNF-alpha, IKBKB, and RELA belong to the NFkB signaling pathway; IFN-gamma to JAK/STAT signaling pathway; GATA3 to the GATA family of Zinc-finger transcription factors, which are involved in transcriptional regulation of T-cell antigen receptor genes, IL-5 gene, and genes involved in adipocytes differentiation; and GADD45G is a member of a family of genes whose transcript levels are increased following stressful growth arrest conditions and treatment with DNA-damaging agents.

These genes were transduced into HUVEC cells using retroviral vectors. Human umbilical vein endothelial cells (HUVEC) were obtained from Clonetics and cultured in EGM containing bovine brain, extract (12 microgram/ml), human epidermal growth factor (10 ng/ml), hydrocortisone (1 microgram/ml), gentamicin (50 microgram/ml), amphotercin-B (50 ng/ml), and 2% fetal bovine serum for 34 passages and sub-cultured with trypsin/EDTA as described by the manufacturer (Clonetics). Experiments were performed by culturing HUVEC in 96-well plates (Nunc), in the presence of various cytokines, activators, for the indicated times.

The retroviral vector used to transfect the HUVEC was derived from the MoMLV-based pFB vector (marketed by Stratagene). Test genes were inserted downstream of the MoMLV LTR. A marker gene, for monitoring the efficiency of gene transfer, was also included in the vector. The marker gene was the truncated form of the human nerve growth factor receptor (NGFR; see Mavilio, 1994, Blood 83:1988), which is separated from the test gene on the vector by an ˜100 bp fragment of the human eIF4G internal ribosomal entry site sequence (IRES; see Gan, 1988, J. Biol. Chem. 273:5006). Other marker genes such as green fluorescent protein (GFP) or beta-galacosidase can also be used. The control vector is the vector without the test gene, containing only the marker gene.

Retroviral vector plasmid DNA was transfected into AmphoPack-293 cells (Clonetech) by the modified calcium phosphate method according to the manufacturer's protocol (MBS transfection kit, Stratagene). Other standard methods for transduction or transfection of cells for expression of genes can also be used.

Cell supernatants were harvested 48 hours post-transfection, filtered to remove cell debris (0.45 micron filter), and transferred onto exponentially growing HUVEC. DEAE dextran (concentration 10 microgram/ml) was added to facilitate vector transduction. After 5-8 hour incubation, the viral supernatant was removed, and the cells were cultured for an additional 40 hours. Gene transfer efficiency was determined by FACS using an NGFR-specific monoclonal antibody and was typically ≧70%. Transduced cells were re-plated into 96-well plates and grown to confluency (2-3 days). Other cells that could be used in this analysis (or in the methods of this invention generally) include primary microvascular endothelial cells, aortic and arteriolar endothelial cells, and endothelial cell lines such as EAhy926 and E6-E7 4-5-2G cells, and human telomerase reverse transcriptase-expressing endothelial cells (for suitable cells, see Simmons, 1992, J. Immunol. 148:267; Rhim, 1998, Carcinogenesis 19:673; and Yang, 1999, J. Biol. Chem. 274:26141).

Expression of ICAM-1 in HUVEC cells was determined by ELISA. The ELISA was conducted as follows. Microtiter plates containing HUVEC were blocked by incubating with 200 μl of 1% Blotto (Pierce Chemical Co.) in PBS for 30 minutes. Plates were washed five times with 0.05% Blotto/PBS between each staining step below. Primary antibodies or isotype control antibodies were added (0.1-2 microgram/ml in 0.05% Blotto/PBS) and incubated for 1 hr. After washing, plates were then incubated with 50 microliter of 1:3000 peroxidase-conjugated anti-mouse IgG (Promega) or biotin-conjugated anti-mouse IgG for 1 hr. After washing, plates were incubated with 1:1000 peroxidase-conjugated streptavidin (Pierce Chemical Co.) in 0.05% Blotto/PBS for 1 hr. Plates were then washed and developed with 100 μliter TMB substrate (Kierkegaard and Perry Laboratories, Gaithersburg, Md.) for 5-10 minutes. The reaction was stopped, and the absorbance (OD) was read at 450 nm (subtracting the background absorbance at 650 nm) with a Molecular Devices plate reader.

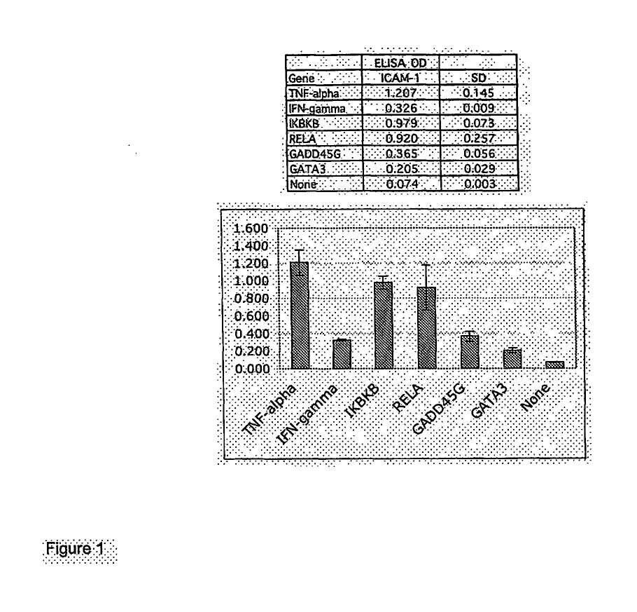

Relative to the control cells, each of the over-expressed genes resulted in a 3 to 16-fold induction of ICAM-1 expression. These results are presented in the table and bar graph in FIG. 1. Because all genes tested resulted in an induction of expression of ICAM-1, the results do not enable one to deduce that the genes tested represent more than one signal transduction pathway. Thus, measurement of a single signal transduction pathway response does not necessarily enable one to group gene products into a common pathway.

B. Grouping Genes and Gene Products into Common Signal Transduction Pathways by Measuring Multiple Responses. By expanding the number of responses to activation (“parameters” or “readouts”), one can identify functionally related genes and gene products to place those genes in a signal transduction pathway. It will be appreciated that the definition of functional relation by such a method is different from other methods in the art, in that the method does not rely on nucleotide or protein sequence homology, the presence of common protein domains, sub-cellular localization (e.g. soluble, trans-membrane, or nuclear), enzymatic activity (as defined in biochemical assays), or direct protein-protein interaction. Instead, the definition of functional relation provided by the method arises from the observation that two genes, when over-expressed in a cell, activate an identical or very similar set of parameters (such as genes that are activated by the over-expressed gene).

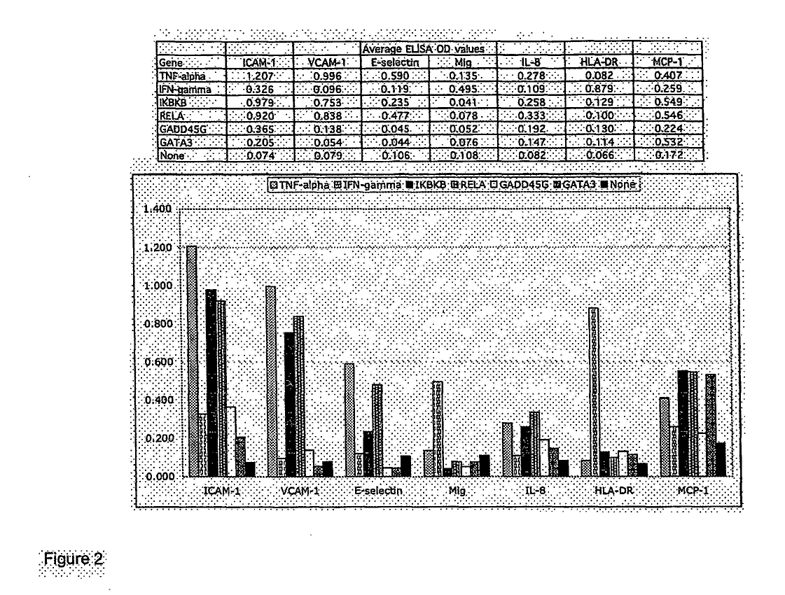

This method can be illustrated simply by expanding the parameters in the test system described in Example 1.A. from ICAM-1 to include VCAM-1, E-selectin, MIG, IL-8, HLA-DR, and MCP-1. Each of these parameters can be measured by ELISA assays known in the art. The results obtained from such ELISA assays are presented and analyzed as shown in FIG. 2 and Table 1. FIG. 2 shows the average ELISA values measured in these assays for ICAM-1, VCAM-1, E-selectin, MIG, IL-8, HLA-DR, and MCP-1 in a table and bar graph.

The results show that the response to gene over-expression of each of the additional genes or parameters is unique and distinct from the response observed for ICAM-1. For-example, IFN-gamma activates ICAM-1, MIG and HLA-DR; GATA3 activates ICAM-1 and MCP-1; and RELA activates ICAM-1, VCAM-1, E-selectin, IL-8 and MCP-1.

The present invention also provides computer-assisted methods for analyzing the data collected in pathway analysis. For data storage and retrieval, one can employ a suitable database, such as an Oracle-based database, where data sets are stored along with all the associated experimental information (genes, compounds, cells, lots, dates, and the like). Desired capabilities include data storage, retrieval, export to text or flat files, and data visualization. To address the inherent variability of biological systems, the present invention provides an envelope method for determining significance of change in parameter level induced by gene over-expression relative to control.

In one embodiment of this method, two sets of replicates of the control “empty” vector (no gene) are placed on each plate. The ELISA OD data from each set are averaged, providing two points for estimating the variability of the control for a given readout. The averaging of the replicates is employed so that the effect of any outliers is reduced. For a given readout, the two points are then divided by the overall average, and the log of the ratio is then calculated, thus providing an estimate of the deviation of the control from the mean value. This operation centers the data obtained from each experiment and helps remove any bias introduced by any potential difference in the OD level of the control. Such deviation curves of the control are collected from many experiments, and the overall average of these curves then constitutes the zero (control) profile.

In the next step, an envelope is formed by connecting the one-standard deviation points for each readout. The envelope is expanded outwards, parallel to its original position, by the same amount above and below the zero profile, until the deviation profiles (e.g. 95% confidence) are completely within the upper and lower limits. This constitutes the prediction envelope at a defined (e.g. 95%) confidence level. The deviation curves for control samples in all tests are expected to fall within the limits of the envelope; otherwise, the test is disqualified. Profiles obtained through gene over-expression are tested against this envelope. The gene-specific profile is “centered” by obtaining the log of its ratio to the values of the control. This log-ratio profile is said to be significantly different than control at a defined significance level (e.g. 95%) if one of the parameters falls outside the limits of the appropriate envelope. The assays described herein are of sufficient throughput to generate multiple repeat experiments rapidly, and the result of repeated experiments greatly improves data quality and enhances statistical significance of the observations. In one embodiment, all the samples are done in triplicate, and tests are repeated multiple times as well.

Table 1 shows the results of a statistical analysis, using Pearson's correlation coefficient, of the sets of numerical values (average ELISA OD values for all readouts), as presented in FIG. 2, obtained for each test gene compared to each other. Mean ELISA OD values for each parameter were calculated from triplicate samples per experiment. Mean values were then used to generate ratios between treated and matched control (e.g. media, DMSO, empty vector-transduced) parameter values within each experiment. These normalized parameter ratios were then log₁₀ transformed. Log expression ratios were used in all Pearson correlation calculations. Pearson correlation was done in Partek.

TABLE 1

| | Pairwise comparison (Pearson's correlation coefficient) | | | | | | |
|---|---|---|---|---|---|---|---|
| | TNF-alpha | IFN-gamma | IKBKB | RELA | GADD45G | GATA3 | None |
| TNF-alpha | 1.000 | | | | | | |
| IFN-gamma | −0.507 | 1.000 | | | | | |
| IKBKB | 0.918 | −0.400 | 1.000 | | | | |
| RELA | 0.964 | −0.593 | 0.958 | 1.000 | | | |
| GADD45G | 0.566 | −0.091 | 0.785 | 0.627 | 1.000 | | |
| GATA3 | −0.034 | −0.045 | 0.299 | 0.178 | 0.519 | 1.000 | |
| None | −0.209 | −0.240 | −0.050 | −0.031 | −0.058 | 0.765 | 1.000 |

A statistically significant correlation (>0.9, shaded cell in Table 1) is observed for the TNF-alpha, IKBKB and RELA genes. These genes are all members of the NFkB signaling pathway. Thus, by comparing expression profiles of readouts in cells over-expressing test genes, one can group genes into common signaling pathways. Individual cell signaling pathways do not exist in isolation but are connected and depend on other signaling pathways. Indeed, methods for identifying such pathway relationships and the intersections between pathways are also provided by the present invention, as illustrated in the following Example, which demonstrates that the methods of the invention can be applied to identify the interactions, and points of interaction, between two different signaling pathways.

### EXAMPLE 2

### Identifying Interactions Between Signal Transduction Pathways

This Example illustrates how the methods of the invention can be used to group genes into common signal transduction pathways and to identify signal transduction pathways that interact with one another and the component(s) that mediate such interaction. In part A, a set of genes is compared and subsets grouped into distinct signal transduction pathways, and in part B, interactions between the pathways, and the components that mediate such interactions are identified.

A. Grouping Genes into Signal Transduction Pathways by Gene Over-Expression. Genes encoding key elements of pro-inflammatory pathways (IL-1, TNF-alpha, CD40, and IFN-gamma), the core NFkB pathway, the PI3K/Akt pathway, and the RAS/RAF/MEK pathway (see Table 2) were introduced into endothelial cells by retroviral transduction and allowed to express their encoded proteins for 48 hours, substantially as described in Example 1. The gene-modified endothelial cells were then subjected to 24 hour cytokine stimulation.

TABLE 2
Over-expressed genes

| Gene | Gene description | GenBank no. |
|---|---|---|
| TNFRSF1A | TNF-alpha receptor type I | BC010140 |
| RIPK1 | Receptor-interacting serine threonine kinase 1 (RIP) | NM_003804 |
| TNFRSF5 | CD40 | BC012419 |
| TNFB | TNF-β (lymphotoxin A) | D12614 |
| TNFRSF10B | TRAIL receptor 2 | BC001281 |
| TNFA | TNF-alpha | NM_000594 |
| IKBKB* | I-κB kinase β(IKKB), constitutively active | AF031416 |
| RELA | NF-κB subunit 3 (p65) | NM_021975 |
| IRAK1 | IL-1 receptor-associated kinase 1 | BC014963 |
| MGC3067 | Hypothetical protein MGC3067 | BC002457 |
| MEK1* | MAP2K1, constitutively active R4F | NM_002755 |
| MEK2* | MAP2K2, constitutively active K71W | L11285 |
| RAF* | Raf1, constitutively active | L00212 |
| RAS* | H-Ras, constitutively active V12 | NM_005343 |
| MYD88 | Myeloid differentiation primary response gene 88 | NM_002468 |
| SHP2* | Phosphotyrosyl-protein phosphatase (SH-PTP2), | L03535 |
| | dominant negative | |
| LSM1 | Sm-like protein 1 (CASM) | BC001767 |
| IFNG | IFN-gamma | NM_000619 |
| MHC2TA | MHC class II transactivator (C2TA) | NM_000246 |
| P2Y6R | Pyrimidinergic receptor P2Y | BC000571 |
| TRADD | TNFR1-associated death domain protein | BC004491 |
| IL11RA | IL-11 receptor alpha | BC003110 |
| AKT1* | AKT1-estrogen receptor fusion, constitutively active upon | BC000479 |
| | tamoxifen treatment | |
| PI3K* | p110 subunit of pI3K, constitutively active | M93252 |

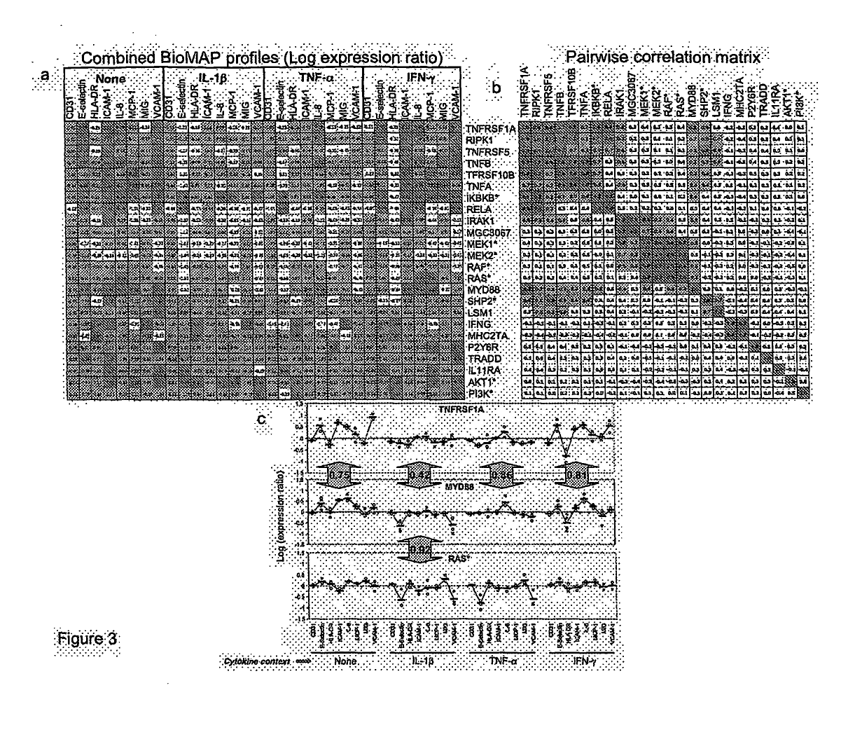

Gene over-expression generally results in activation of the target pathway (in contrast to most pharmaceutical drugs, which are typically inhibitors). Gene over-expression effects were examined in four parallel systems comprising endothelial cells incubated with IL-1-beta, with TNF-alpha, with IFN-gamma, or with media alone (recombinant human IFN-gamma, TNF-alpha, and IL-1-beta were obtained from R&D Systems (Minneapolis, Minn.). Eight parameters (CD31, E-selectin, HLA-DR, ICAM-1, IL-8, MCP-1, MIG and VCAM-1) were evaluated in each system by ELISA, using methodology substantially as described in Example 1. FIG. 3, part a, shows average mean log parameter expression ratios from two to four individual experiments in each system, while FIG. 3, part b, shows pairwise correlations of experiments across all four systems (using 28 data points/gene for calculating the Pearson correlation: E-selectin, HLA-DR, ICAM-1, IL-8, MCP-1, MIG and VCAM-1 readouts across four systems).

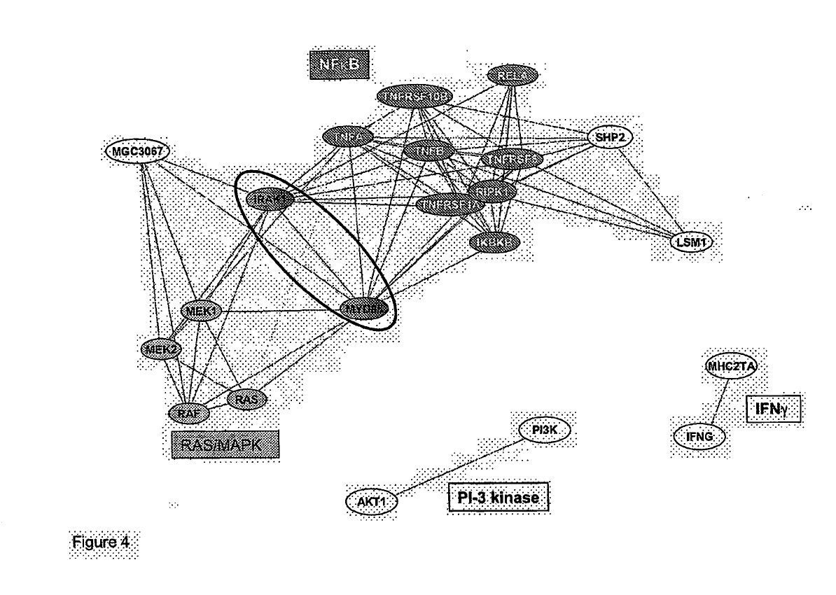

Strikingly, the highest functional correlations observed were between genes whose products carry out the same function (e.g., MEK1* and MEK2* r=0.91, or TNFA and TNFB r=0.86; see FIG. 3, part b) or genes that are members of a common pathway. For example, TNFRSF5 (CD40), TNFA (TNF-alpha) and TNFB (TNF-beta), and the TNFRSF1A (TNF-alpha receptor type I) all stimulate the NF-κB pathway, whose intracellular signaling components include RIPK1, IKBKB*, and RELA. The pairwise correlation of experiments reveals that genes that are known to be members of the same pathway show functional similarity as assessed by statistically significant correlation coefficients in these assays. Together, these NFκB pathway genes comprise a well-defined cluster shown in FIG. 4, a two-dimensional representation of the pairwise correlation matrix from FIG. 3, part b. Similarly, RAS/MAPK pathway members RAS*, RAF*, MEK1* and MEK2* are functionally similar, and comprise a distinct cluster. PI3K and its downstream partner AKTI*, and IFN-gamma and the MHC2TA transcription factor it induces, also define discrete and well separated functional units. Thus, these signature gene response profiles, assessed across multiple systems, reveal participation of gene products in common cell-signaling pathways.

Multi-system BioMAP analysis described here is also capable of identifying novel participants in signaling pathways and defining their network interactions. For example, the intracellular phosphatase SHP2 is known to have a role in growth factor-induced signaling (You et al. (2001) J. Exp. Med. 193, 101-110). In our experiments, however, SHP2* showed clear functional similarity to members of the NFkB pathway, for example up-regulation of ICAM-1 and VCAM-1 in control cells, and down-regulation of HLA-DR in IFN-g-treated cells, indicating that this protein can regulate NFkB signaling in endothelial cells. In fibroblasts, SHP2 has indeed been shown to interact physically with the NFkB complex and is required for the NFkB-dependent production of IL-6. Similarly, our studies reveal similarity of function of the hypothetical protein MGC3067 to IRAK1, MEK1 and MEK, suggesting that it plays a role in the RAS/MAPK pathway.

B. Similarity of Function Reveals Interactions between Signaling Pathways. Even more strikingly, multi-system analysis can reveal novel routes by which pathways can interact. As shown in FIG. 4, MYD88 and IRAK1 were functionally related to genes encoding members of both the NFkB and RAS/MAPK pathways, suggesting that MYD88 and IRAK1 can interact with both of these pathways.

To explore this observation further, we re-examined the response to MYD88 and genes encoding representative members of the RAS/MAPK and NFkB pathways (RAS* and TNFRSF1A, respectively) in all individual cell systems. As shown in FIG. 3 part c, over-expression of MYD88 and TNFRSF1A increased E-selectin, ICAM-1, IL-8 and VCAM-1 levels in IFN-gamma-treated and control endothelial cells, consistent with the known ability of MYD88 and TNFRSF1A to activate the NFkB pathway. By contrast, the response induced by MYD88 in IL-1-beta-treated cells was similar to that induced by RAS*, the main effect being to inhibit expression of the adhesion molecules VCAM-1 and E-selectin. Over-expression of MYD88 thus appears to stimulate the RAS/MAPK pathway under these conditions. Blocking the RAS/MAPK pathway by treatment with the MEK inhibitor PD098059 reversed the effect of MYD88 or RAS* over-expression, confirming that the effects induced by both genes were mediated by the RAS/MAPK pathway. MYD88 (and IRAK1) are known to be involved in IL-1-induced but not in TNF-induced signaling, and PD098059 indeed had no effect on VCAM-1 expression in TNF-alpha-treated cells. Multi-system analysis can thus detect novel functional interrelationships between different signaling pathways.

Combined these examples show that functional profiles generated by gene over-expression in multiple parallel cell systems can be used to cluster genes into signaling pathways, discover pathway interactions and identify pathway members that are involved in these pathway-pathway interactions.

While the analytical methods for classification of genes into signaling pathways by hierarchical clustering techniques described in this example and Example 1 are functional, other more sophisticated approaches for the analysis of pathway interactions can also be used. Such methods, which have been used successfully to mine large microarray datasets can be adapted to the methods of the present invention, and include both supervised and unsupervised methods. Unsupervised methods, which include a variety of clustering methods (Hierarchical clustering, k-means, Gene Shaving), can identify patterns in the data and create meaningful groupings of the genes based on some similarity of the gene over-expression profiles. Supervised methods of the invention (Tree Harvesting, Neural Networks, Support Vector Machines) allow the discovery of correlations between an outcome and key explanatory variables. These methods can be trained to produce predictive models for characterizing new data. Once the appropriate statistical methods have been applied to the data, the analyzed data and resulting predictive models can be included in a database, increasing the ability of the database to assign genes into signaling pathways accurately and also predict biological function of unknown genes.

FIG. 3, part b shows results from a method of the invention used to identify potential connectivity between genes based on their over-expression profiles. In this method, a pairwise similarity matrix is constructed for all the genes that have been identified having profiles significantly different than zero. With a permutation technique, an average distribution of the correlation coefficients that will be obtained by chance is constructed and the values of the correlations that correspond to a required level of significance are obtained. The original similarity matrix is filtered using these values, thus providing a consistent way of identifying correlations coefficients with potential biological significance. This method also allows calculation of a false positive rate, providing the user with a way of balancing “hit rate” and stringency of correlation significance. This method confirmed significance of correlations among members of NFkB, RAS/MAPK, PI3K/AKT and IFN-gamma signaling pathways as well as correlation of MYD88 and IRAK1 with RAS/MAPK pathway genes (FIG. 3, part b, correlation values passing the described statistical significance test are shaded in gray). Only significant correlations surviving the permutation test are used to generate two-dimensional maps (as in FIG. 4) allowing the user to focus on fewer, but potentially more biologically relevant, correlations

Other components to facilitate high throughput gene screening in accordance with the methods of the present invention include the development of templates for automated entry of gene names and plate locations from external files as well as interfaces for public gene and protein databases, such as GenBank, OMIM, PubMed, and ExPASy.

C. Grouping Genes into Signal Transduction Pathways by Gene Knock-Down. SiRNAs targeting genes encoding members of the core IFN-gamma driven JAK/STAT pathway, signal activator and tranducer 1 (STAT1), IFN-gamma receptor 2 (IFNGR2), and Janus Kinase 1 (JAK1), as well as siRNAs directed against a number of known genes from other signaling pathways were introduced into HUVEC cells, and the expression of readout parameters across a number of stimulatory conditions was measured, as described in Example 2A. Early passage (<5) exponentially growing HUVEC cells were harvested, washed once with PBS, and resuspended at 2×10⁶ cells in 100 microliter Nucelofection solution (Human Umbilical Vein Endothelial Cell Nucleofector Kit, AMAXA, Koeln, Germany). SiRNA (15 microliter of a 20 micromolar solution; Dharmacon, Lafayette, Colo.) was added to the cell suspension, transferred into an electroporation cuvette, and electroporated using the U-1 setting on an AMAXA Nucleofector device. The cell suspension was then transferred into a separate tube containing 3 ml of complete EGM-2 media (Clonetics), incubated at 37° C. for 10 minutes, and plated into 96-well microtiter plates (25,000 cells/well) for cytokine activation and ELISA analysis as described above. Statistical analyses and pairwise correlation analyses of functional profiles obtained by gene knock-down were performed in the manner as described for the gene-over-expression approach described above.

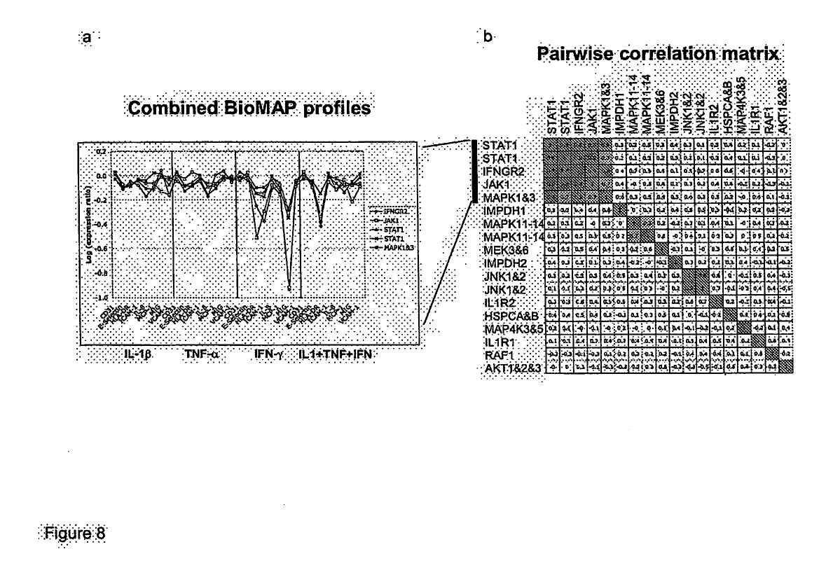

FIG. 8 shows that the highest functional correlation is indeed between the genes that are members of a same signaling pathway, for example STAT1, JAK1 and IFNGR2 genes are members of the IFN-gamma driven JAK/STAT pathway. In addition, one can identify novel functional associations between gene products. For example, MAPK1 (ERK2) and MAPK3 (ERK1) have not been previously implicated to play a role in the JAK/STAT pathway, in fact MAPK1 and MAPK3 are members of a growth factor-driven MAP kinase signaling pathway. Data presented here indicate that MAPK1 and/or MAPK3 genes are a connection point between JAK/STAT and MAP kinase signaling pathway. Thus, by measuring effects of gene knock-down across multiple systems, followed by statistical analysis and pairwise comparison of resulting functional profiles one can identify novel functional associations between components of different signaling pathways, and establish links between these pathways.

These results demonstrate that the functional effects of individual genes, and the functional relationships between effects of different genes, depend in large part on the complex system or network in which they act. Combining data from multiple systems allows enhanced precision in separating and clustering genes by function, and additional insights can arise from comparing responses within each of several different complex systems in which particular combinations of signaling pathways are active. The system dependence of gene function homologies (as shown for MYD88 and RAS* genes) illustrates the critical importance of evaluating gene (and drug effects) across multiple cellular systems, as provided by the present invention, designed to embody a broad range of cell- and environment-dependent system behaviors.

The multiplexed activity profiling in multiple parallel cellular systems described here is both scalable and amenable to automation, thus having the potential to characterize pathways (and mechanisms of action) of novel genes or biologically active molecules rapidly through “similarity of function’ with activities of known drugs and compounds. Such assay of gene and drug function across multiple complex systems permits a novel, discovery science approach to cell biology. Applications include large scale gene function screening and classification; integration of biology and pathophysiology into target validation and drug development to improve the efficiency of drug, development programs; and large scale characterization and analysis of environment and cell differentiation-dependent biological responses.

Thus, these methods of the invention can be used to group genes into common signal transduction pathways and to identify the points of interaction between two different signal transduction pathways. The methods cannot however predict order of the components in a signaling pathway or the directional flow of a signal in the pathway. Such ordering of signaling pathway components is achieved using methods described in the following example. Thus, the present invention provides a set of methods for the comprehensive analysis of signal transduction pathways.

### EXAMPLE 3

### Ordering of Components in a Signal Transduction Pathway

In this Example, a variety of inhibitors and activators are applied in accordance with the present methods to deduce the order of components in a signal transduction pathway.

In this method of the invention, activators, including gene over-expression, and inhibitors, including chemical compound inhibitors, are used to order the components of a signal transduction pathway. To illustrate the method, the signal transduction pathway components identified in Example 1.B. as belonging to the same signaling pathway, TNF-alpha, IKBKB, and RELA (known to be in the NFkB signaling pathway) are ordered. It should be noted that, while nucleotide and protein sequence analysis could be used to predict that TNF-alpha is a soluble protein, IKBKB is a kinase, and RELA is a transcription factor, such analysis could not be used to predict whether RELA activates IKBKB or vice versa or whether RELA activates TNF-alpha or vice versa.

In practicing the method of the present invention, one first needs to activate the various components in the pathway to be ordered. In this illustrative embodiment, the over-expressing cell lines described in Example 1.A. and 1.B can be employed. One also selects the readout to be measured, and again, Example 1.B. shows that over-expression of the TNF-alpha, IKBKB; or RELA genes induces VCAM-1 expression in HUVEC cells, so VCAM-1 expression can be selected as the readout for this illustrative application of the method.

One next selects the inhibitors to be employed in the method. Because it is important to appreciate that the method can take advantage of known inhibitors of a pathway, including specific inhibitors of a pathway component, but is not limited to the use of either known or specific inhibitors, the method will be illustrated in two steps. The first step shows the results obtained using only a known inhibitor of the NFkB pathway.

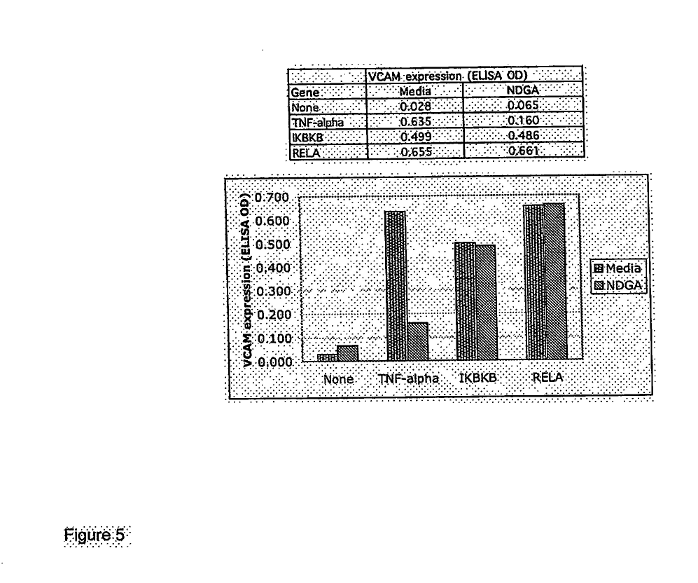

The inhibitor selected for this first illustrative step was NDGA (nordihydroguaiaretic acid), a known inhibitor of the NFkB pathway (see van Puijenbroek et al., February 1999, Cytokine 11(2):104-110). NDGA was thus applied to the cell lines over-expressing one of the three pathway components, TNF-alpha, IKBKB, and RELA, and to a control cell line, and VCAM-1 expression was measured by ELISA, as described in Example 1B. The results are shown in the table and bar graph in FIG. 5. The results demonstrate that NDGA will inhibit TNF-alpha induced VCAM-1 expression, but not IKBKB or RELA induced VCAM-1 expression. Thus, TNF-alpha is upstream in the pathway from IKBKB and RELA.

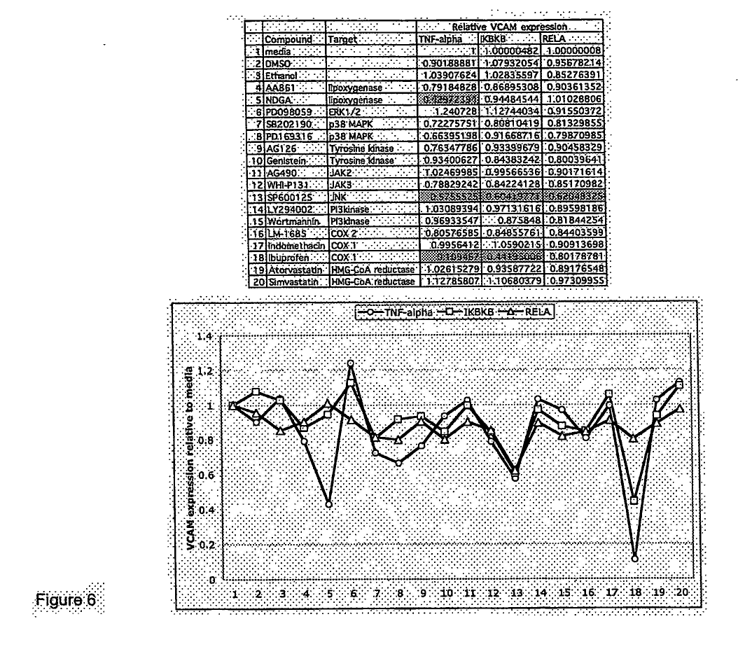

In the next illustrative step, a larger panel of drugs and drug-like compounds is employed to identify inhibitors that act downstream or upstream from all test genes. FIG. 6 shows the panel of drugs tested and the effect of each on VCAM-1 expression (as measured by ELISA) in the HUVEC cell lines over-expressing one of the three pathway component genes TNF-alpha, IKBKB, and RELA in both a table and a linear plot (the number on the x axis corresponds to the drug number in the table). Among all the drugs tested, three compounds can inhibit either of the three test genes TNF-alpha, IKBKB, or RELA. These compounds are NDGA, ibuprofen, and SP600125. NDGA inhibits only the TNF-alpha gene, ibuprofen inhibits TNF-alpha and IKBKB genes, and SP600125 inhibits all three (TNF-alpha, IKBKB and RELA) genes.

Because NDGA inhibits the pathway only in TNF-alpha over-expressing cells, the IKBKB and RELA genes must be downstream of TNF-alpha in the signaling pathway; otherwise, over-expression of those genes would not be insensitive to the inhibitory effect of NDGA. Similarly, because ibuprofen inhibits the pathway in both TNF-alpha and IKBKB over-expressing cells, but not in RELA over-expressing cells, and because of the results obtained with NDGA, the IKBKB gene must be upstream of the RELA gene. Thus, based on these two inhibitors, one can deduce that the order of genes in the signaling pathway is TNF-alpha is upstream of IKBKB in the pathway, and IKBKB is upstream of RELA in the pathway. The result observed with the SP600125 inhibitor confirms the deduction.

This illustrative step also demonstrates that indirect inhibitors can be useful in the method. The pharmacological inhibitors used in the method do not have to be specific for the over-expressed (or otherwise activated) genes. Thus, specific inhibitors of other pathways that interact with a signaling pathway of interest can be “indirect” or “non-specific” inhibitors of the signaling pathway of interest. Those of skill in the art will appreciate in this regard that none of the inhibitors used in this Example 3 is a specific NFkB signaling pathway inhibitor. The primary target for NDGA is 5-lipoxygenase; for ibuprofen, cyclooxygenases 1 and 2; and for SP600125, stress-activated Jun kinase (JNK). Moreover, and as noted above, in addition to direct and indirect pharmacologic inhibitors, other inhibitors of gene function can be used in the method as well, including but not limited to antisense DNA or RNA, siRNA, dominant negative mutants, inhibitory peptides, and the like.

This Example demonstrates that even without any knowledge of the components of a signaling pathway, the methods of the invention alone can identify and arrange genes that belong to that signaling pathway. When the pathway genes are unknown, one can, in first step group genes into common pathways based on the similarity of profiles generated by over-expressing those genes in multiple parallel systems and then measuring a panel of readouts. Once the genes are grouped in a pathway, one can order those genes in the pathway by exposing a panel of cells that consists of members that over-express each gene to be ordered to a number of pharmacological inhibitors of gene function. Using inhibitors that act downstream of each test gene in the signaling pathway, one can identify the order of the genes in the pathway by analysis of the inhibition profile obtained. The fewer the genes inhibited by an inhibitor, the higher up (or closer to the beginning) of the pathway those few genes can be placed.

Those of skill in the art will appreciate that the present invention can be applied to map all of the signaling pathways in any cell of any origin. While human endothelial cells were employed in this illustrative embodiment of the invention, any cell type can be used to practice the present invention, different human cell lines (e.g. HeLa, Jurkat, and the like) and human primary cell types (fibroblasts, T cells, smooth muscle cells, and the like), as well as cells from non-human mammals and from other eukaryotes, such as plants, insects, and yeast. The invention can be practiced with two or more genes that can be activated (for example, by over-expression or use of a promoter trap) and two or more inhibitors at least one of which, in the simplest case of two genes, inhibits only a single gene.

While there are myriad applications of this aspect of the invention, two aspects merit additional attention. First, the invention can be used to define signaling pathways and order their components functionally. In this application, the invention may often be practiced in a mode in which novel members of known signaling pathways as well as new signaling pathways are identified by clustering genes in a set into pathways. Second, the invention can be used to characterize drugs and potential drug candidates, thereby identifying new uses for drugs or off-target activities, including those that may cause unwanted side effects, thus providing new methods for treating disease with drugs. For example, in the illustrative embodiment of the invention discussed above, the COX inhibitor ibuprofen was demonstrated to inhibit the NFκB pathway downstream of IKBKB.

Ultimately, if one employs specific over-expression or otherwise activated profiles for all human genes, the present invention could be practiced to group all of those genes into all of the signaling pathways as shown in Example 1; to identify the interactions, and points of interactions, between those pathways, as shown in Example 2; and to order the genes in each pathway, as shown in Example 3.

### EXAMPLE 4

### Characterizing the Mechanism of Action of a Compound by Drug Treatment of Gene-Over-Expressing Cells

The pathway information developed by practice of the methods of the present invention facilitates the in-depth characterization (mechanism-of-action studies) of chemical compounds. As shown in Example 3, the profiles induced by gene over-expression can be inhibited by compounds that act on the over-expressed gene itself or downstream in the pathway. For example, the profiles induced by RAS*, RAF* or MEK1* genes are affected by MEK inhibitors PB098059 and Uo126 but not by inhibitors that act on other signaling pathways, such as p38MAPK inhibitors (PD169316, SB202190), JAK inhibitors (AG490, WHI-P131) and others. A high throughput approach can be used to test a compound against genes from known signaling pathways, as well as genes of unknown pathway origin.

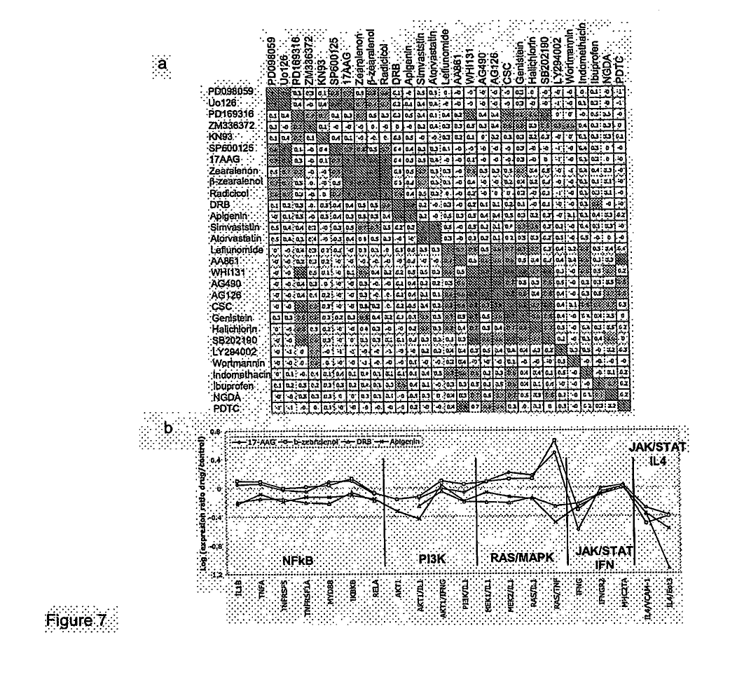

The usefulness of this approach for precise mapping of the effects of a compound on cellular signaling pathways is shown in FIG. 7. Twenty-nine compounds with known or unknown molecular targets were screened in 20 assays, each over-expressing a single gene from the NFkB, PI3K/Akt, RAS/MAPK or JAK/STAT signaling pathways (JAK/STAT IFN-gamma and JAK/STAT IL-4). The resulting profiles were correlated using the permutation method described above. Statistically significant correlations presented in FIG. 7, part a, are indicated by shading (dark grey for correlation coefficients in the range of 0.75 to 1, and light grey for the range of 0.55 to 0.75). As expected, compounds that inhibit same molecular targets cluster together in these assays, for example MEK inhibitors (PB09059 and Uo126, r=0.90), HMG-CoA inhibitors (simvastatin and atorvaststin, r=0.84) and Hsp90 inhibitors (17-AAG and radicicol, r=0.96).

Most interestingly, unexpected correlations were observed, for example between Hsp90 inhibitors (17-AAG, radicicol), and mycotoxins with estrogen-like properties (beta-zearalenol, zearalenone). Profiles for 17-MG, and beta-zearalenol are shown in FIG. 7, part b and are highly correlated across all 20 gene assays (r=0.90). This indicates that targets for 17-MG and beta-zearalenol are either the same or a part of the same protein complex where inhibition of either component induces similar biology. 17-AAG is an Hsp90 inhibitor, while beta-zearalenol and zearalenone bind to estrogen receptors alpha and beta. Hsp90 is a chaperone that forms a complex with and is critical for functioning of the estrogen receptor complex. Thus, functional mapping of drug effects using methods described here has identified a functional link between Hsp90 and estrogen receptor, and implicated Hsp90 as a potential target for blocking estrogen receptor signaling. Analysis of responses of sets of genes from individual pathways to drug treatment provides further insight into drug activities. As shown in FIG. 7, part b, functional profiles of casein kinase 2 inhibitors DRB and apigenin overlap with those of 17-MG and beta-zearalenol only in the JAK/STAT portion of the overall profile. Hsp90 is known to play a role in stabilizing casein kinase 2 complex. Casein kinase 2 phosphorylates estrogen receptor on position serine 167, and this phosphorylation is critical for transactivation activity of the estrogen receptor. Thus, the data presented here confirm known links between casein kinase 2, estrogen receptor and Hsp90 chaperone, and also reveal a new role for casein kinase 2 and estrogen receptor in the regulation of JAK/STAT pathways. As the number of genes that actively read out in such assays is expanded, one can more precisely map drug activities and, ultimately, be able to predict the molecular target(s) for any compound.

Thus, the present invention provides assays for compound profiling as well as a variety of reagents and protocols for gene over-expression and drug treatment that can be packaged individually or in various combinations and marketed in kit form. Such reagents include reagents and protocols for, the large-scale production of retrovirus vectors, quality control, arraying into 96-well format deep-well plates, and storage. Sets of gene reagents, where each set constitutes a functionally similar group (aka functional components of a signaling pathway), are also provided by the invention. For such analyses one can use either the full set of over-expression systems, or a smaller set of selected parameters/conditions that strongly respond to gene over-expression. The smaller parameter set will facilitate higher throughput initial testing, which can then be followed by more complete analyses. All of the steps in compound profiling can be automated, allowing for rapid mapping of a compound's effects on a large number of genes/pathways. Applications of this technology include identification of molecular targets for those compounds for which the exact cellular target is not known, as well as for discovery of secondary cellular targets (off-target activity) for compounds that have been developed against known targets. Assays can also be used for screening and drug discovery in a way that is different from standard screening approaches where chemical libraries are generally screened in one-target single-parameter assays. The present invention provides that one would use a panel of over-expression systems to discover new compounds with biologically interesting profiles in a target-agnostic way.

### EXAMPLE 5

### Characterizing the Mechanism of Action of a Compound by Using Known Gene-Specific Inhibitor

Functional profiles generated by gene under-expression using a gene-specific inhibitor (e.g. siRNA knock-down) can be compared to functional profiles generated by treatment of cells with compounds, and if the profiles match, then one can deduce that the under-expressed gene product is the target for the compound; or the under-expressed gene product is a part of a signaling pathway and is located in the pathway near the compound target (most often just upstream or downstream); or the under-expressed gene product is a part of a protein complex, where one member of such a protein complex is targeted by the compound, and the other member is under-expressed gene product and disruption of any component of such a protein complex (either by compound or gene knock-down) results in a similar phenotype (functional profile).

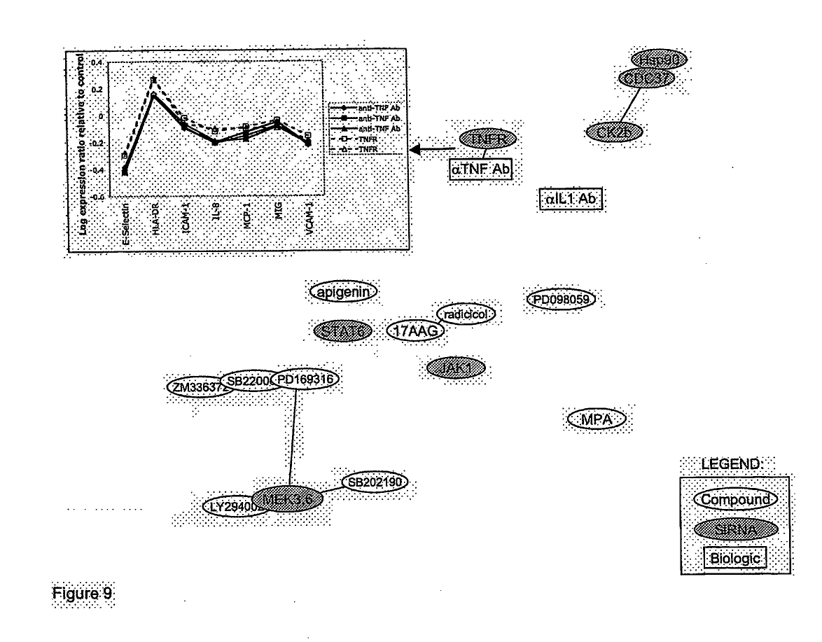

This is illustrated in the example in FIG. 9 which shows a two-dimensional presentation of the pairwise correlation matrix for functional profiles generated by treatment of cells with compounds or biologics or by siRNA-mediated gene knock-down. The cells used to generate functional profiles were HUVEC stimulated with a mixture of cytokines IL-1-beta, TNF-alpha and IFN-gamma, and readout parameters were as described in Example 2A. Agents with similar mechanism of action induce similar functional profiles and are positioned near each other in space and connected by lines (which indicate that the correlation is statistically significant). For example, the anti-TNF-alpha antibody (anti-TNF-Ab) and the siRNA (TNFR) directed against TNF-alpha receptor type I (aka TNFRSF1A) induce similar functional profiles (see box showing multiple repeats of profiles), and therefore cluster in this two-dimensional map. Furthermore, functional profile induced by siRNA-mediated dual knock-down of kinases MEK3 and 6 is similar to those induced by p38MAPKinase inhibitors e.g. SB202190 and PD169316. MEK3 and MEK6 are part of the MAPkinase signaling pathway involved in inflammatory response, and are main activators of p38MAPkinases (there are four isoforms of p38MAPK). Thus, blocking an immediate activator of p38MAPKinases has the same functional consequence as inhibiting p38MAPkinases themselves. These results have two implications. They link MEK3 and 6 with p38MAPKinase (for pathway mapping purpose), and implicate MEK3 and MEK6 as potential targets for blocking p38MAPKinase signaling pathway.

In the further example shown in FIG. 9, functional profiles induced by siRNA knock-down of casein kinase 2 beta (CK2b), Cdc37, and Hsp90 gene expression are functionally similar. This is of particular interest because these proteins form a multi-protein complex, and thus affecting any of the individual components of a complex leads to similar functional phenotype. We can conclude that functional profiling methods described in the present invention can also identify proteins that potentially form multi-protein complexes.

Combined, these examples show broad applicability of the methods described in the present invention for discovery and characterization of signaling pathways and signaling pathway components, and for determination of mechanism of action of compounds and biologics. The present invention, having been described in detail and illustrated by example above, will be understood by those of skill in the art, in light of the patent applications, patents, and scientific journal reference cited herein, all of which are incorporated herein by reference, to be embodied by the claims that follow.

1. A method for determining components of a signaling pathway, the method comprising:
exposing a set of recombinant cells to at least one factor that activates or inhibits said signaling pathway, wherein each member of said set of recombinant cells either over or under-expresses a gene to be determined as a component in said pathway;
recording changes in at least two different cellular parameter readouts after exposure to said at least one factor;
deriving a functional profile from changes in said parameter readouts for each said gene to be determined as a component in said pathway;
and clustering genes in pathways according to similarities in functional profile.

2. A method for determining the presence of an interaction between a first and a second signaling pathway, the method comprising:
exposing a set of recombinant cells to at least one factor that activates or inhibits at least one said signaling pathway, wherein each member of said set of recombinant cells either over or under-expresses a gene that is a component in at least one said signaling pathway;
recording changes in at least two different cellular parameter readouts after exposure to said at least one factor;
deriving a functional profile from changes in said parameter readouts for each said gene;
determining if said over or under-expressed gene in one of said pathways responds to said activators in a manner that correlates to the responses measured for one of said over or under-expressed genes in the other of said pathways, wherein if such a correlation exists then said first and said second signaling pathways interact, and the common component of said interaction is the gene for which said correlation was observed.

3. A method for ordering the components of a signaling pathway, the method comprising:
exposing a first set of recombinant cells to a first inhibitor of said signaling pathway, wherein each member of said set of recombinant cells either over or under-expresses a gene that is a component in said signaling pathway, wherein said signaling pathway is activated;
exposing a second set of said recombinant cells to a second inhibitor of said signaling pathway, wherein each member of said set of recombinant cells either over or under-expresses a gene that is a component in said signaling pathway;
recording changes in at least two different cellular parameter readouts after exposure to said at said first and said second inhibitors;
determining the epistatic relationships between said components and said inhibitors of said pathway; and correlating the relative order of action of said inhibitors with the order of the components of the pathway.

4. A method for determining the mechanism of action for a test compound on a signaling pathway, the method comprising:
exposing a set of recombinant cells, each member of which over or under-expresses a target gene, to a test compound;
recording changes in at least two different cellular parameter readouts after exposure to said at least one factor;
deriving a functional profile from changes in said parameter readouts for each said gene; and
comparing said functional profile with functional profiles of a set of control compounds having known mechanisms of action; and
determining if said test compound produces a functional profile comparable to one or more of said control compounds.

5. The method according to claim 4, wherein said exposing is performed in the presence of at least one factor that activates or inhibits at least one signaling pathway.

6. The method according to claim 4, wherein said set of recombinant cells under-expresses a target gene as a result of exposure to an agent that specifically inhibits expression of said target gene.

7. The method according to claim 1, wherein said exposing step comprises exposing said set of cells to at least two different factors.

8. The method according to claim 1, wherein said exposing step comprises exposing said set of cells to at least three different factors.

9. The method according to claim 1, wherein changes in at least four parameters and not more than ten parameters are recorded.

10. The method according to claim 1, wherein functional profiles are ordered in a correlation plot by multidimensional scaling.
